(19) United States
(12) Patent Application Publication
(10) Pub. No.: US 2009/0124680 A1
(43) Pub. Date: May 14, 2009

(54) USE OF PRODRUG COMPOSITION CONTAINING NAPHTHOQUINONE-BASED COMPOUND FOR MANUFACTURE OF MEDICAMENT FOR TREATMENT OR PREVENTION OF DISEASES INVOLVING METABOLIC SYNDROME

(75) Inventors: Sang-Ku YOO, Gwacheon-si (KR); Sang Woo YOO, Seoul (KR); Ku Suk KANG, Siheung-si (KR)

Correspondence Address: BIRCH STEWART KOLASCH & BIRCH, PO BOX 747, FALLS CHURCH, VA, 22040-0747, US

(73) Assignees: Mazence Inc., Daejeon (KR); KT&G Corporation, Daejeon (KR)

(21) Appl. No.: 12/261,932

(22) Filed: Oct. 30, 2008

(30) Foreign Application Priority Data
Oct. 31, 2007 (KR) 10-2007-0110041

(51) Int. Cl. A61K 31/4178; C07D 311/80; C07D 405/12; C07D 307/92

(52) U.S. Cl. 514/397; 549/388; 548/311.4; 549/458

(57) ABSTRACT

Provided is a use of a prodrug composition containing a naphthoquinone-based compound of Formula 1 for the manufacture of a medicament for treatment or prevention of metabolic syndrome diseases.

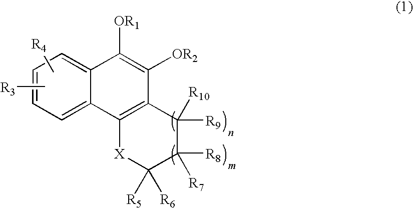

wherein R₁ to R₁₀, X, m and n are as defined in the specification.

### FIELD OF THE INVENTION

The present invention relates to a use of a prodrug composition containing a naphthoquinone-based compound for the manufacture of a medicament for treatment or prevention of metabolic syndrome diseases.

### BACKGROUND OF THE INVENTION

Metabolic syndromes refer to diseases accompanied by health risk factors such as hypertriglyceridemia, hypertension, glycometabolism disorders, blood coagulation disorders and obesity. According to the ATP III criteria of the National Cholesterol Education Program (NCEP) published in 2001, individuals are diagnosed with the metabolic syndrome by the presence of three or more of the following components: 1) A waistline of 40 inches (102 cm) or more for men and 35 inches (88 cm) or more for women (central obesity as measured by waist circumference), 2) A triglyceride level above 150 mg/dL, 3) A high density lipoprotein (HDL) level less than 40 mg/dL (men) or under 50 mg/dL (women), 4) A blood pressure of 130/85 mmHg or higher, and 5) A fasting blood glucose level greater than 110 mg/dL.

At present, the most effective way to ameliorate or fight against the conditions associated with such metabolic syndromes is known to be getting more exercise and dietary control, and losing weight. All of the currently effective ways of fighting against the metabolic syndromes have in common the fact that they facilitate energy metabolism, thus resulting in promoted expenditure of surplus energy in the body leading to prevention of energy accumulation. Effective expenditure of such surplus energy is considered a method for treating the metabolic syndromes. Promoting energy metabolism is most important for effective elimination of surplus energy. For this purpose, it is essential to achieve inhibition of lipogenesis, inhibition of gluconeogenesis, facilitation of glucose consumption, facilitation of fat oxidation, and collective activation of factors involved in biogenesis and activation of mitochondria which is a central apparatus of energy metabolism.

There is yet little known about targets to treat the metabolic syndrome diseases, whereas numerous target proteins or genes are known only for treating individual diseases and therefore there have been proposed some methods for the prevention or treatment of such diseases via use of the above-mentioned corresponding target proteins or genes. However, there is still a room for further significant improvement even in treatment of individual diseases such as metabolic syndromes including obesity, diabetes and the like. In spite of the fact that a great deal of studies has been conducted on treatment of diseases, there are yet no drugs available for the treatment of various diseases resulting from excess energy intake and aging.

Most of diseases including obesity, diabetes, metabolic syndromes, degenerative diseases and mitochondrial dysfunction-related diseases, i.e., large numbers of diseases including metabolic syndrome diseases, stem from imbalance of energy metabolism and oxidation-reduction state. For this reason, the present invention has also employed a method of confirming the presence/absence of activation effects of candidate compounds on AMP-activated protein kinase (AMPK), as the most fundamental primary test to confirm biological efficacy of compounds of interest on metabolic syndrome diseases.

Meanwhile, once AMPK is activated, a variety of physiological events are consequently affected in the downstream of the mechanism thereof. In this regard, factors to be regulated and expression phenomena are provided as follows.

### 1. Glycometabolism

In muscle tissues and myocardium, AMPK promotes muscle contraction and thereby facilitates intake of glucose. That is, AMPK activates GLUT 1, or induces migration of GLUT 4 to a plasma membrane, regardless of insulin action, resulting in increased glucose uptake into cells (Arch. Biochem. Biophys. 380, 347-352, 2000, J. Appl. Physiol. 91, 1073-1083, 2001). After increasing glucose uptake into cells, AMPK activates hexokinase, thereby increasing flux of glycometabolism processes and simultaneously inhibiting glycogen synthesis. It is known that in myocardial tissues under ischemic conditions, AMPK activates a phosphorylation process of 6-phosphofructo-2-kinase (PFK-2), with consequent activation of a metabolic cascade leading to increased flux of glycometabolism (Curr. Biol. 10, 1247-1255, 2000). In addition, it was confirmed that activation of AMPK in the liver inhibits release of glucose from hepatocytes, and activity of phosphoenolpyruvate carboxykinase (PEPCK) and glucose-6-phosphatase, which are gluconeogenesis enzymes, is inhibited by AMPK (Diabetes 49, 896-903, 2000). This is because AMPK independently takes part in regulation of a blood glucose level via inhibition of release of glucose from the liver, irrespective of insulin.

### 2. Fat Metabolism Regulation and AMPK

Upon reviewing a mechanism of AMPK participating in fat metabolism, AMPK induces phosphorylation of acetyl-CoA carboxylase, thereby resulting in inhibition of fatty acid synthesis. Therefore, AMPK is known to facilitate fatty acid oxidation, by the action of decreasing an intracellular concentration of malonyl-CoA that is an intermediate of fatty acid synthesis and is an inhibitor of carnitine palmitoyl-CoA transferase I (CPT I). CPT I is an enzyme essential for a fatty acid oxidation process wherein fatty acids enter mitochondria and are oxidized, and is known under the control of malonyl-CoA. In addition, AMPK is known to inhibit activity of HMG-CoA reductase and glycerol phosphate acyl transferase (GPAT), involved in synthesis of cholesterol and triacylglycerol, through phosphorylation (J. Biol. Chem. 277, 32571-32577, 2002, J. Appl. Physiol. 92, 2475-2482, 2002).

Meanwhile, it was found that activation of AMPK in the liver inhibits the activity of pyruvate kinase, fatty acid synthase and ACC through phosphorylation of carbohydrate-response-element-binding protein (ChREBP) (J. Biol. Chem. 277, 3829-3835, 2002). In addition, activity of sterol-regulatory-element binding protein-1 (SREBP-1), which plays an important role in differentiation of adipocytes, is also inhibited by the action of AMPK, which results then in inhibition of adipocyte differentiation.

### 3. Protein Synthesis Regulation and AMPK

In the protein synthesis process, AMPK inhibits synthesis of proteins via inhibition of mTOR and p70S6K by activating TSC, or AMPK inhibits translation elongation via activation of elongation factor-2 (eEF2) kinase and inactivation of eEF2 through phosphorylation thereof. It was found that eEF2 kinase is a direct substrate for AMPK (J. Biol. Chem. 278, 41970-41976, 2003).

As discussed above, AMPK is known to play a central role in energy metabolism of glucose, protein, fat and the like, in vitro and in vivo. Neil, et al (Nature Drug Discovery, 3 (April), 340, 2004) has asserted that AMPK and Malonyl-CoA are possible targets for the treatment of metabolic syndromes, and they have also stated that patients suffering from metabolic syndromes can be characterized by insulin resistance, obesity, hypertension, dyslipidemia, dysfunction of pancreatic beta cells, type II diabetes and manifestation of arteriosclerosis. It was hypothesized that a common feature linking these multiple abnormalities is dysregulation of AMPK/Malonyl-CoA energy level-sensing and signaling network. It was proposed that such dysregulation leads to alterations in cellular fatty acid metabolism which in turn cause abnormal fat accumulation, cellular dysfunction and ultimately diseases. Evidence is also presented that factors activating AMPK and/or reducing malonyl-CoA levels might reverse these abnormalities and syndromes or prevent incidence of these diseases.

Roger, et al (Cell, 117, 145-151, 2004) have suggested that AMPK may be a possible target to control obesity by lowering activity of hypothalamic AMPK, thereby increasing a content of malonyl-CoA and then regulating appetite for food intake.

Lee, et al (Nature Medicine, 13 (June), 2004) have suggested that alpha-lipoic acid can exert anti-obesity effects by suppressing hypothalamic AMPK activity, thus controlling appetite. They have also reported that alpha-lipoic acid promotes fat metabolism via activation of AMPK in muscle tissues, not in the hypothalamus, and alpha-lipoic acid is therapeutically effective for the treatment of obesity because it facilitates energy expenditure by activating UCP-1, particularly in adipocytes.

Diraison, et al (Diabetes 53, S84-91, 2004) have reported that activation of AMPK in pancreatic cells leads to four-fold increases in expression of the gut hormone peptide YY responsible for appetite control and thus appetite can be regulated by the action of AMPK in tissues other than the hypothalamus.

Nandakumar, et al (Progress in Lipid Research 42, 238-256, 2003) have proposed that, in ischemic heart diseases, AMPK would be a target to treat ischemia reperfusion injuries via regulation of fat and glucose metabolism.

Min, et al (Am. J. Physiol. Gastrointest Liver Physiol 287, G1-6, 2004) have reported that AMPK is effective for regulation of alcoholic fatty liver.

Genevieve, et al (J, Biol. Chem. 279, 20767-74, 2004) have reported that activation of AMPK inhibits activity of an iNOS enzyme that is an inflammation mediator in chronic inflammatory conditions or endotoxin shock, including obesity-related diabetes and thus AMPK will be effective for developing new medicines having a mechanism capable of enhancing insulin sensitivity. In addition, they have reported that inhibition of iNOS activity is effected by activation of AMPK, and thus this finding is clinically applicable to diseases such as septicemia, multiple sclerosis, myocardial infarction, inflammatory bowel diseases and pancreatic beta-cell dysfunction.

Zhi-ping et al (FEBS Letters 443, 285-289, 1999) have reported that endothelial NO synthase (eNOS) is phosphorylated by AMPK, in the presence of Ca-calmodulin in murine muscle cells and myocardial cells. This represents that AMPK is implicated in heart diseases including angina pectoris.

Javier, et al (Genes & Develop. 2004) have reported that a lifespan can be extended by limiting utilization of energy and such a prolonged lifespan is achieved in a manner that an in vivo AMP/ATP ratio is increased and therefore the α2 subunit of AMPK is activated by AMP. Therefore, they have suggested that AMPK may function as a sensor to detect the relationship between lifespan extension and energy level and insulin-like signal information.

Based on the above-mentioned action mechanisms of AMPK, the present invention provides a use of a prodrug composition containing a naphthoquinone-based compound, as an effective therapeutic agent for the treatment of metabolic syndrome diseases.

Meanwhile, the naphthoquinone-based compound does not exert therapeutic effects until it is absorbed into the body in an amount exceeding a certain concentration. However, such a naphthoquinone compound drug is highly insoluble in an aqueous solution. For this reason, the naphthoquinone-based compounds suffer from various difficulties associated with formulation of preparations for in vivo administration, in spite of their excellent pharmacological effects.

More specifically, the naphthoquinone-based compounds are sparingly-soluble materials which are soluble at a low degree of about 2 to 10% only in high-solubility solvents, such as CH₂Cl₂, CHCl₃, CH₂ClCH₂Cl, CH₃CCl₃, Monoglyme, and Diglyme, but are poorly soluble in other ordinary polar or non-polar solvents. Therefore, when these drug compounds are administered per se or in the form of a conventional simple formulation, there is substantially no absorption of the drug into the body, that is, the bioavailability of the drug is very low, which makes it impossible to exert the intrinsic efficacy of the drug. To this end, in order to sufficiently and satisfactorily exploit inherent pharmacological properties of these naphthoquinone-based drugs, there is an urgent need for development and introduction of a novel technique which is capable of maximizing the bioavailability of these drug compounds.

Bioavailability is the degree to which a drug becomes available to the target tissue after administration. Many factors can affect the bioavailability. For example, the drug bioavailability may vary depending upon the dosage form and dissolution rate of the drug. Poor bioavailability is a significant problem encountered in the development of pharmaceutical compositions. Low water-solubility, i.e., poor solubility, means that a drug of interest has solubility of 10 to 15 mg/mL, severely 1 mg/mL and therefore tends to be cleared from the gastrointestinal tract before being absorbed into the circulatory system.

In order to exhibit desired therapeutic effects following absorption of the drug into the body, the drug should be dissolved in digestive organs or otherwise should be finely divided similar to such a dissolved state and then smoothly absorbed into the body. However, unlike water-soluble drugs which are readily dissolved in water, sparingly-soluble drugs which are poorly dissolved in water exhibit no solubility or very low solubility in digestive organs, which inevitably results in deterioration of bioavailability of the drug. For this reason, a great deal of research has been focused on development of a preparation to promote the in vivo absorption of sparingly-soluble drugs. Currently, there is a strong need for development of a formulation technique which is capable of solving the problem of poor solubility of an active drug ingredient in a pharmaceutical composition containing a naphthoquinone compound as a therapeutically active ingredient and is capable of achieving high in vivo bioavailability.

### SUMMARY OF THE INVENTION

Therefore, the present invention has been made to solve the above problems and other technical problems that have yet to be resolved.

Therefore, an object of the present invention is to provide a use of a prodrug composition comprising a naphthoquinone-based compound, for the manufacture of a medicament for treatment or prevention of metabolic syndrome diseases. For this purpose, the composition of the present invention is capable of achieving increased water solubility of the naphthoquinone-based compound which is effective as a therapeutic agent for the treatment of metabolic syndrome diseases but is poorly soluble in water, improved bioactivity of the naphthoquinone-based compound upon in vivo administration thereof, and additionally enhanced bioavailability of an active ingredient.

In accordance with an aspect of the present invention, the above and other objects can be accomplished by the provision of a use of a prodrug composition comprising a naphthoquinone-based compound represented by Formula 1 below, for the manufacture of a medicament for treatment or prevention of metabolic syndrome diseases:

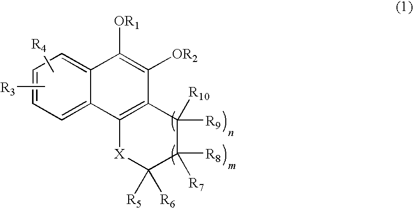

wherein,

A) X is selected from the group consisting of C, N, O and S;

B) R₁ and R₂ are each independently —SO₃—Na⁺, or a substituent represented by Formula 2 below or a salt thereof:

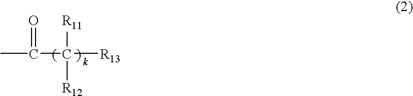

wherein

R₁₁ and R₁₂ are each independently hydrogen or substituted or unsubstituted, linear or branched C₁-C₂₀ alkyl;

R₁₃ is selected from the group consisting of substituents i) to viii):

i) hydrogen;

ii) substituted or unsubstituted, linear or branched C₁-C₂₀ alkyl;

iii) substituted or unsubstituted amine;

iv) substituted or unsubstituted C₃-C₁₀ cycloalkyl or C₃-C₁₀ heterocycloalkyl;

v) substituted or unsubstituted C₄-C₁₀ aryl or C₄-C₁₀ heteroaryl;

vi) —(CRR′—NR″CO)₁—R₁₄ wherein R, R′ and R″ are each independently hydrogen or substituted or unsubstituted, linear or branched C₁-C₂₀ alkyl, R₁₄ is selected from the group consisting of hydrogen, substituted or unsubstituted amine, cycloalkyl, heterocycloalkyl, aryl and heteroaryl, and 1 is in the range of 1 to 5;

vii) substituted or unsubstituted carboxyl; and

viii) —OSO₃—Na⁺; and

k is in the range of 0 to 20, provided that when k is 0, R₁₁, and R₁₂ are absent and R₁₃ is directly bonded to a carbonyl group;

C) R₃ and R₄ are each independently hydrogen, halogen, alkoxy, hydroxy or C₁-C₆ alkyl, or R₃ and R₄ may be taken together to form a cyclic structure;

D) R₅, R₆, R₇, R₈, R₉, and R₁₀ are each independently selected from the group consisting of hydrogen, hydroxy, and C₁-C₂₀ alkyl, C₁-C₂₀ alkene, C₁-C₂₀ alkoxy, C₃-C₁₀ cycloalkyl, C₃-C₁₀ heterocycloalkyl, C₄-C₁₀ aryl, C₄-C₁₀ heteroaryl and any combination thereof, any of which groups being substituted or unsubstituted, or two of R₅ to R₁₀ may be taken together to form a cyclic structure; and

E) m and n are each independently 0 or 1, provided that when either of m and n is 0, carbon atoms adjacent to m or n may form a cyclic structure via a direct bond.

The aforesaid prodrug composition can improve solubility, in vivo stability and pharmacokinetic properties of a naphthoquinone-based compound which is poorly insoluble.

That is, in the prodrug composition of a naphthoquinone-based compound, substituent(s) on the carbonyl group may be appropriately selected within the above-defined range, depending on a variety of factors such as solubility, stability, bioavailability and delivery or absorption system of target drugs, As a consequence, the activity of the in vivo-administered naphthoquinone-based compound will increase.

Hence, when the prodrug composition is used in the treatment or prevention of metabolic syndrome diseases, it is possible to effectively obtain desired therapeutic effects even with administration of a trace amount of the naphthoquinone-based compound, due to improved solubility and activity thereof.

As used herein, the term “prodrug” means an agent that is converted into the parent drug in vivo. The prodrug in the context of the present invention is intended to encompass compounds defined by formula 1 as well as pharmaceutically acceptable salts and isomers thereof.

The term “pharmaceutically acceptable salt” means a formulation of a compound that does not cause significant irritation to an organism to which it is administered and does not abrogate the biological activity and properties of the compound. Examples of the pharmaceutical salt may include acid addition salts of the compound with acids capable of forming a non-toxic acid addition salt containing pharmaceutically acceptable anions, for example, inorganic acids such as hydrochloric acid, sulfuric acid, nitric acid, phosphoric acid, hydrobromic acid and hydroiodic acid; organic carboxylic acids such as tartaric acid, formic acid, citric acid, acetic acid, trichloroacetic acid, trifluoroacetic acid, gluconic acid, benzoic acid, lactic acid, fumaric acid, maleic acid and salicylic acid; or sulfonic acids such as methanesulfonic acid, ethanesulfonic acid, benzenesulfonic acid and p-toluenesulfonic acid. Specifically, examples of pharmaceutically acceptable carboxylic acid salts include salts with alkali metals or alkaline earth metals such as lithium, sodium, potassium, calcium and magnesium, salts with amino acids such as lysine, arginine and guanidine, and salts with organic bases such as dicyclohexylamine, N-methyl-D-glucamine, tris(hydroxymethyl)methylamine, diethanolamine, choline and triethylamine. The compound in accordance with the present invention may be converted into a salt thereof, by conventional methods known in the art.

The term “isomer” means a compound of the present invention or a salt thereof that has the same chemical formula or molecular formula but is optically or sterically different therefrom.

The term “alkyl” means an aliphatic hydrocarbon group. The alkyl moiety may be a “saturated alkyl” group, which means that it does not contain any alkene or alkyne moiety. The alkyl moiety may also be an “unsaturated alkyl” group, which means that it contains at least one alkene or alkyne moiety. The alkyl may contain 1 to 20 carbon atoms. Preferably, the allyl may be a C₁-C₆ lower alkyl.

The alkyl group may be substituted or unsubstituted. Typical examples of the alkyl group may include, but are not limited to, methyl, ethyl, propyl, isopropyl, butyl, isobutyl, t-butyl, pentyl, hexyl, ethenyl, propenyl, butenyl, cyclopropyl, cyclobutyl, cyclopentyl, and cyclohexyl. When substituted, the alkyl group may be substituted with one or more groups which are individually and independently selected.

The term “aryl” refers to an aromatic group which has at least one ring having a conjugated pi (π) electron system and includes carbocyclic aryl (for example, phenyl) and heterocyclic aryl (for example, pyridine) groups. This term is intended to include monocyclic or fused-ring polycyclic (i.e., rings which share adjacent pairs of carbon atoms) groups.

The term “heteroaryl” refers to an aryl group that contains at least one heterocyclic ring.

Examples of aryl or heteroaryl may include, but are not limited to, phenyl, furan, pyran, pyridyl, pyrimidyl and triazyl.

The term “heterocycloalkyl” means a carbocyclic group in which one or more ring carbon atoms are substituted with oxygen, nitrogen or sulfur and which includes, for example, but is not limited to furan, thiophene, pyrrole, pyrroline, pyrrolidine, oxazole, thiazole, imidazole, imidazoline, imidazolidine, pyrazole, pyrazoline, pyrazolidine, isothiazole, triazole, thiadiazole, pyran, pyridine, piperidine, morpholine, thiomorpholine, pyridazine, pyrimidine, pyrazine, piperazine and triazine.

R₃, R₄, R₅, R₆, R₇, R₈, R₉ and R₁₀ in Formula 1 may be optionally substituted. When substituted, the substituent group(s) is (are) one or more group(s) individually and independently selected from cycloalkyl, aryl, heteroaryl, heteroalicyclic, hydroxy, alkoxy, aryloxy, mercapto, alkylthio, arylthio, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, trihalomethanesulfonyl, amino including mono- and di-substituted amino, and protected derivatives thereof.

The term “composition” as used herein means a mixture of the naphthoquinone-based compound with other chemical components, such as diluents or carriers. The composition facilitates in vivo administration of the compound to a subject organism. The composition can also be obtained by reacting compounds of interest with acids such as hydrochloric acid, hydrobromic acid, sulfuric acid, nitric acid, phosphoric acid, methanesulfonic acid, p-toluenesulfonic acid, salicylic acid and the like.

The substituent R₂ may be preferably a structure that is capable of minimizing steric hindrance whereby the reactivity of a molecule is affected by spatial disposition of atomic groups between R₁ and R₂. In one preferred embodiment of the present invention, R₂ may be a structure of —CO—(CH₂)₀₋₂₀—R₁₄ wherein R₁₄ may be selected from the group consisting of hydrogen, hydroxy, carboxyl, SH, C₄-C₁₀ aryl, C₄-C₁₀ heteroaryl, C₃-C₁₀ cycloalkyl, C₃-C₁₀ heterocycloalkyl, amine and any combination thereof.

Preferably, k is in the range of 0 to 3. When k is 0, R₁₁ and R₁₂ are absent, so properties of the prodrug composition may be determined by R₁₃.

R₁₃ may be appropriately selected depending on kinds of desired drug delivery targets.

In one preferred embodiment of the present invention, R₁ or R₂ may be a substituent of Formula 3:

—CO—(CRR′—NR″CO)ᵢ—R₁₄ (3)

wherein R, R′ and R″ are each independently hydrogen or substituted or unsubstituted, linear or branched C₁-C₂₀ alkyl or R and R″ may be taken together to form a cyclic structure, R₁₄ may be selected from the group consisting of hydrogen, substituted or unsubstituted C₁-C₁₀ alkyl, substituted or unsubstituted C₁-C₁₀ alkoxy, substituted or unsubstituted amine, substituted or unsubstituted C₃-C₁₀ cycloalkyl, heterocycloalkyl, aryl and heteroaryl, and i is in the range of 1 to 3. The substituted amine may be preferably C₁-C₆ alkyl-substituted amine.

Such a substituent structure is a structure that substantially contains an amino acid group. Examples of the amino acid may include glycine, alanine, valine, leucine, isoleucine, phenylalanine, tryptophan, methionine, serine, threonine, cysteine, tyrosine, asparagine, glutamine, aspartic acid, glutamic acid, lysine, arginine, proline, histidine, etc. Particularly preferred is glycine, alanine, valine, proline, isoleucine or histidine.

The amino acid may be an alpha-amino acid side chain where an amino group and a carboxyl group are attached to the same carbon, or may be an amino acid of alpha carbon and alpha amino group side chain. Therefore, the amino acid and the naphthoquinone-based compound can be linked to each other via the carboxyl group of the alpha-amino acid.

In one preferred embodiment of the present invention, R₁₃ may be carboxyl, more preferably carboxylic acid, for example malonic acid, succinic acid, or nicotinic acid.

When R₁₃ is substituted carboxyl, the substituent may be linear or branched C₁-C₂₀ alkyl which may be optionally substituted with one or more substituents selected from the group consisting of hydrogen, hydroxy, carboxyl, C₄-C₁₀ aryl and C₄-C₁₀ heteroaryl.

Specific examples of R₁₃ may include, but are not limited to, —COOH, —CH₂—COOH, —(CH₂)₂COOH, —OCO—C₁-C₁₁ alkyl, —OCO—CO—C₆ alkyl-C₄-C₁₀ aryl, —OCO—C₀-C₆ alkyl-C₄-C₁₀ aryl-C₄-C₁₀ heteroaryl, —OCO—C₀-C₆ alkyl-C₄-C₁₀ heteroaryl, —OCO—C₀-C₆ alkyl-COOH, and —OCO—C₁-C₆ alkyl-OH.

When the alkyl group is substituted, the substituent may be preferably selected from the group consisting of hydroxy, SH, amine and any combination thereof.

When the aryl or heteroaryl group is substituted, the substituent may be preferably selected from the group consisting of amine, C₁-C₆ alkyl, C₄-C₁₀ aryl, C₄-C₁₀ heteroaryl, C₃-C₁₀ cycloalkyl, C₃-C₁₀ heterocycloalkyl and any combination thereof. More preferably, the aryl or heteroaryl group may be selected from the group consisting of pyridine, aryl or heteroaryl substituted with C₁-C₆ alkyl, and aryl or heteroaryl substituted with C₁-C₆ alkyl and/or aryl.

Examples of the substituted aryl and heteroaryl may include, but are not limited to, C₁-C₆ alkyl-C₄-C₁₀ aryl, C₁-C₆ alkyl-C₄-C₁₀ heteroaryl, C₄-C₁₀ aryl-C₄-C₁₀ heteroaryl, and C₁-C₆ alkyl-C₄-C₁₀ aryl-C₄-C₁₀ heteroaryl.

When cycloalkyl or heterocycloalkyl is substituted, examples of the substituent may preferably include the following groups.

First, when cycloalkyl or heterocycloalkyl is substituted, the substituent may be preferably selected from the group consisting of hydroxy, C₁-C₆ alkyl, C₁-C₆ alkoxy, C₁-C₆ aryl, C₄-C₁₀ heteroaryl, C₃-C₁₀ cycloalkyl, C₃-C₁₀ heterocycloalkyl and any combination thereof. Particularly preferably, the heterocycloalkyl may be selected from the group consisting of piperidine, piperazine and pyrrolidine which may be optionally substituted with C₁-C₆ alkyl.

Ring carbon atoms in the heterocycloalkyl or heteroaryl are substituted with one or more hetero atoms selected from the group consisting of N, S and O.

The above-mentioned prodrug compositions allow exertion of a maximum in vivo efficacy of the naphthoquinone-based compound while not exhibiting detrimental effects on the human body and pharmacological effects of the naphthoquinone-based compound. Therefore, it is expected that the composition of the present invention, comprising the naphthoquinone-based compound as an active ingredient, can improve the bioavailability of the active drug ingredient for treatment of metabolic syndrome diseases. In addition, it is possible to prepare a medicament for effective prevention or treatment of metabolic syndrome diseases.

In Formula 1, X may be selected from the group consisting of C, N, O and S. X is preferably O. Further, the composition in accordance with the present invention comprises a prodrug compound of Formula 1 wherein m is 1 and n is 1 or m is 1 and n is 0. For example, an active ingredient of the prodrug composition may be a compound of Formula 4 or 5 below.

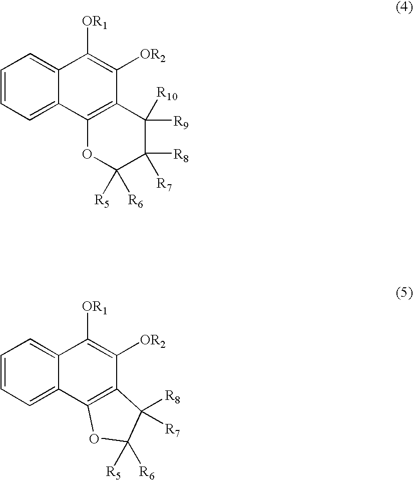

When m is 1 and n is 0, R₃, R₄ and R₆ may be independently hydrogen, or R₃, R₄ and R₅ may be independently hydrogen. Preferred is a prodrug compound of Formula 6 or 7 below.

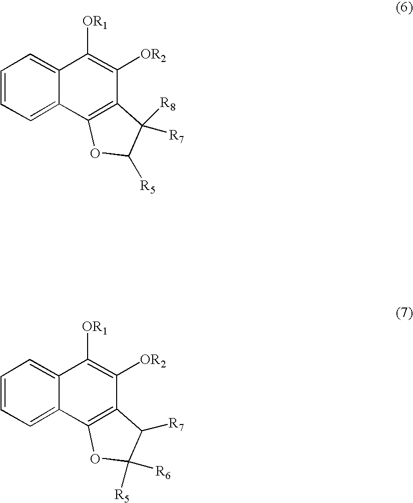

When m is 1 and n is 1, R₃, R₄, R₇, R₈, R₉ and R₁₀ may be independently hydrogen. Preferred is a prodrug compound of Formula 8 below.

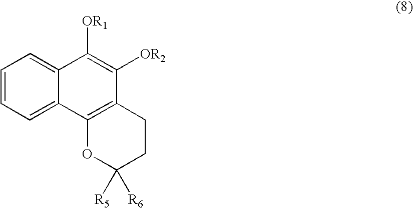

There is no particular limit to the method for preparing a prodrug which is an active ingredient of the prodrug composition, so it can be appropriately prepared by a conventional method known in the art. For example, 1,2-naphthoquinone among compounds of Formula 1 is reduced into 1,2-naphthohydroquinone using zinc (Zn) metal, followed by induction of first acetylation and then second acetylation to thereby synthesize the prodrug compound.

That is, as shown in Reaction Scheme 1 below, reduction of a 1,2-naphthoquinone derivative in the presence of Zn results in production of a 1,2-naphthohydroquinone derivative which is then subjected to first acetylation to obtain a mono-acetyl compound. This is followed by another acetylation to finally synthesize a di-acetyl prodrug derivative of the present invention with a relatively high yield. Based on this synthetic scheme, various compounds of Formula 1 may be synthesized as desired. Exemplary methods for preparing active prodrug compounds are set forth in the following Reaction Schemes.

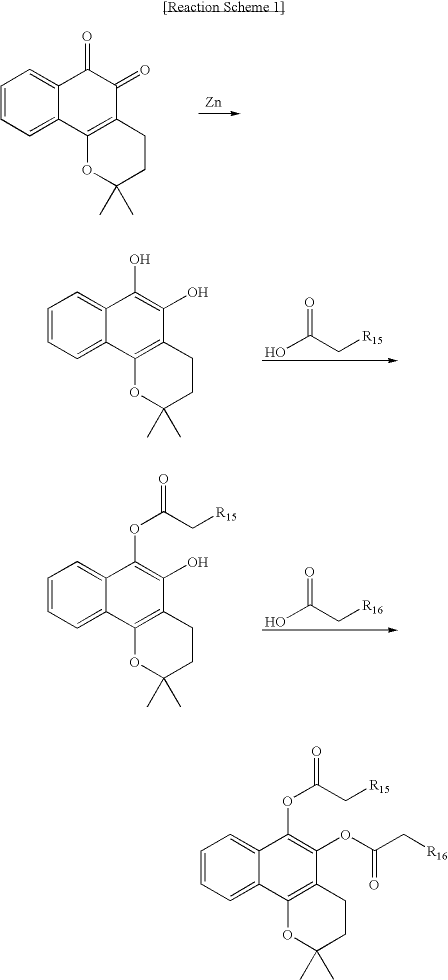

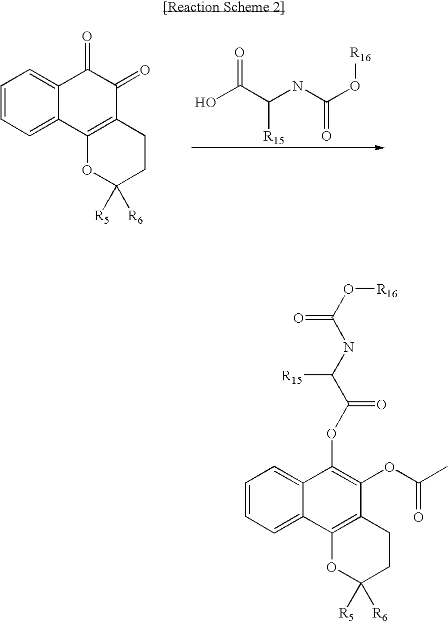

In Reaction Scheme 2, R₅ and R₆ are as defined above, and R₁₅ and R₁₆ are each independently hydrogen, or substituted or unsubstituted, linear or branched C₁-C₂₀ alkyl.

Specifically, when R₁₅ and R₁₆ are different from each other, zinc powder, Na₂S₂O₄, organic acid, triethylamine, HBTU and DMF are added to a naphthoquinone-based compound and the mixture is stirred at room temperature for 10 to 20 hours. EtOAc is added to the reaction mixture which is then filtered and washed with water. Then, the organic extract is dried over Na₂SO₄ and concentrated under reduced pressure to obtain a first acetylated monoacetyl compound which is then directly used without further purification for subsequent second acetylation.

The secondary acetylation is carried out as follows. The first acetylation product is dissolved in acetic anhydride to which a zinc powder catalyst and triethylamine are then added. The mixture is heated, stirred for about 2 hours, and cooled. The solvent is removed under reduced pressure, and the residue is dissolved in EtOAc and washed with water. The organic extract is dried over Na₂S₂O, concentrated under reduced pressure and purified to obtain a desired prodrug compound.

When R₁₅ and R₁₆ are identical to each other, the naphthoquinone-based compound is thoroughly reacted with zinc powder and an excess (2 equivalents or more) of acetic anhydride. The unreacted acetic anhydride is removed under reduced pressure, and the residue is dissolved in CH₂Cl₂ and washed with water. The organic extract is dried over Na₂S₂O, concentrated under reduced pressure and purified to obtain a desired prodrug compound.

The thus-prepared compound may also be obtained as a prodrug in the form of a salt, by hydrolyzing a protecting group of protected amine as set forth in Reaction Scheme 3 below. Specifically, the prodrug prepared according to the procedure of Reaction Scheme 2 is dissolved in 1,4-dioxane to which a solution of an acid (e.g. HCl) in anhydrous 1,4-dioxane is added to elicit hydrolysis. The reaction mixture is stirred at room temperature and dried under reduced pressure to obtain a desired prodrug compound.

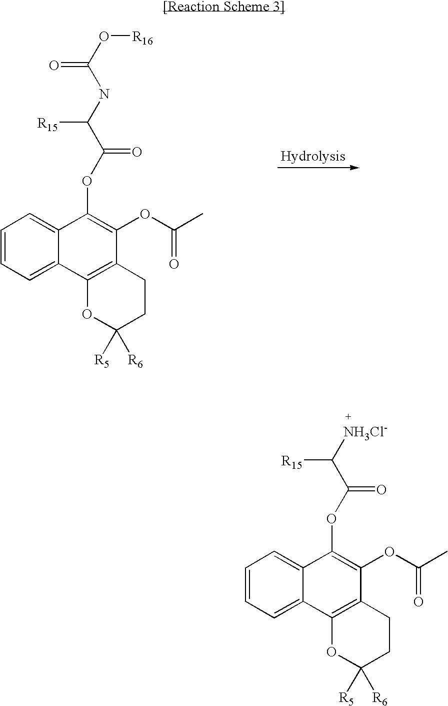

Further, it is also possible to prepare a prodrug containing a ring structure, through the acetylation reaction as above. The chemical reaction procedure is summarized in Reaction Scheme 4 below.

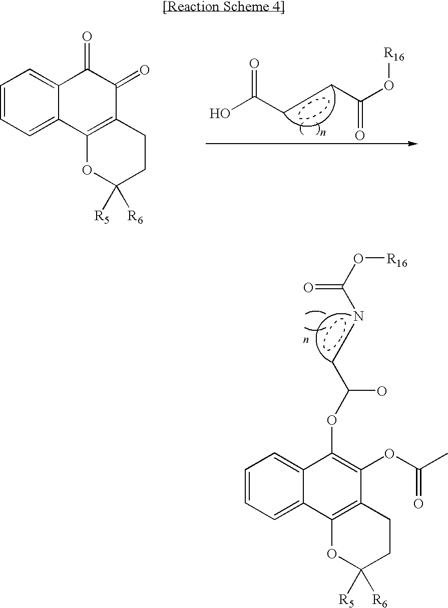

The prodrug prepared according to the procedure of Reaction Scheme 4 may also be prepared as a prodrug in the form of another salt, through acid-mediated hydrolysis as set forth in Reaction Scheme 5 below.

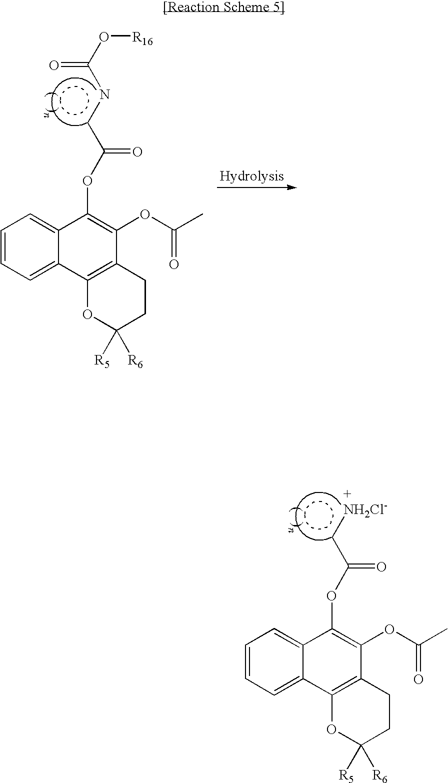

The above-mentioned methods are only exemplary and the invention is not limited thereto. Therefore, it should be understood that various other methods are possible and fall within the scope of the present invention.

In accordance with yet another aspect of the present invention, there is provided a pharmaceutical composition comprising (a) a therapeutically effective amount of the aforesaid prodrug composition: and (b) a pharmaceutically acceptable carrier, diluent or excipient, or any combination thereof.

The term “Pharmaceutical composition” as used herein means a mixture of the aforesaid composition with other chemical components, such as diluents or carriers. The pharmaceutical composition facilitates in vivo administration of the compound to a subject organism. Various techniques of administering a compound are known in the art and include, but are not limited to oral, injection, aerosol, parenteral and topical administrations. The c pharmaceutical composition can also be obtained by reacting compounds of interest with acids such as hydrochloric acid, hydrobromic acid, sulfuric acid, nitric acid, phosphoric acid, methanesulfonic acid, p-toluenesulfonic acid, salicylic acid and the like.

The term “therapeutically effective amount” means an amount of a composition that is effective to relieve or reduce to some extent one or more of the symptoms of the disease in need of treatment, when the composition is administered. Thus, a therapeutically effective amount refers to an amount of the composition which exhibit effects of (i) reversing the rate of progress of a disease; (ii) inhibiting to some extent further progress of the disease; and/or, (iii) relieving to some extent (or, preferably, eliminating) one or more symptoms associated with the disease.

The term “carrier” means a chemical compound that facilitates the incorporation of a compound into cells or tissues. For example, dimethyl sulfoxide (DMSO) is a commonly utilized carrier as it facilitates the uptake of many organic compounds into the cells or tissues of an organism.

The term “diluent” defines a chemical compound diluted in water that will dissolve a composition of interest as well as stabilize the biologically active form of the composition. Salts dissolved in buffered solutions are utilized as diluents in the art. One commonly used buffer solution is phosphate buffered saline (PBS) because it mimics the salt conditions of human body fluid. Since buffer salts can control the pH of a solution at low concentrations, a buffer diluent rarely modifies the biological activity of a compound.

The term “physiologically acceptable” defines a carrier or diluent that is not detrimental to the biological activity and physical properties of the composition.

The compounds used herein may be administered to a human patient per se or in the form of pharmaceutical compositions in which they are mixed with other active ingredients, as in combination therapy, or suitable carriers or excipients. Techniques for formulation and administration of the compounds may be found in Remington's Pharmaceutical Sciences, Mack Publishing Co., Easton, Pa., 18^{th} edition, 1990.

### a) Routes of Administration

Suitable routes of administration may, for example, include oral, intranasal, transmucosal, or intestinal administration; and parenteral delivery, including intramuscular, subcutaneous, intravenous, intramedullary injections, as well as intrathecal, direct intraventricular, intraperitoneal, or intraocular injections.

For example, the active compound may be locally administered in the form of a dip or sustained-release formulation by direct injection of the formulation to solid tumor lesions, not via systemic route. Further, the active compound may also be administered in the form of an antibody-coated liposome as a targeted drug delivery system.

### b) Formulations

The pharmaceutical composition may be manufactured in a manner that is itself known, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in a conventional manner using one or more pharmaceutically acceptable carriers comprising excipients and auxiliaries which facilitate processing of the active compounds into preparations which can be used pharmaceutically. Proper formulation is dependent upon the route of administration chosen. Any of the well-known techniques, carriers, and excipients may be used as is suitable and understood in the art; e.g., in Remington's Pharmaceutical Sciences, supra.

For injection, the agents of the present invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active ingredients with pharmaceutically acceptable carriers well known in the art. Such carriers enable the compounds of the present invention to be formulated as tablet, pill, dragee, capsule, liquid, gel, syrup, slurry, suspension and the like, for oral ingestion by a patient. Pharmaceutical preparations for oral use can be obtained by mixing one or more excipients with one or more compounds of the present invention, optionally grinding the resulting mixture, and processing the mixture of granules, after adding suitable auxiliaries, if desired, to obtain tablets or dragee cores. Suitable excipients may be fillers such as sugars, including lactose, sucrose, mannitol and sorbitol; and cellulose substances such as, for example, corn starch, wheat starch, rice starch, potato starch, gelatin, gum tragacanth, methyl cellulose, hydroxypropylmethyl cellulose, sodium carboxymethyl cellulose, and/or polyvinylpyrrolidone (PVP). If desired, there may be added disintegrating agents such as cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc polyvinyl pyrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally may include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc or magnesium stearate. In soft capsules, the active compounds may be dissolved or dispersed in suitable solvents, such as fatty acid, liquid paraffin, or liquid polyethylene glycols. In addition, stabilizers may also be added. All formulations for oral administration should be in dosage forms suitable for such administration.

For buccal administration, the compositions may take the form of tablets or lozenges formulated in a conventional manner.

For administration by inhalation, the compounds according to the present invention are conventionally delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. Capsules and cartridges of, e.g., gelatin for use in an inhaler or insufflator may be formulated containing a powdered mixture of the compound and a suitable powder base such as lactose or starch.

The compounds may also be formulated for parenteral administration by injection, e.g., by bolus injection or continuous infusion. Formulations for injection may be presented in unit dosage forms, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active ingredients in water-soluble form. Additionally, suspensions of the active ingredients may be prepared in the form of appropriate oily injection suspensions. Examples of suitable lipophilic solvents or vehicles include fatty oils such as sesame oil, or synthetic fatty acid esters such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for reconstitution with a suitable vehicle, e.g., sterile pyrogen-free water, prior to use.

A pharmaceutical carrier for hydrophobic compositions is a co-solvent system comprising benzyl alcohol, a nonpolar surfactant, a water-miscible organic polymer, and an aqueous phase. The co-solvent system may be a VPD co-solvent system. VPD is a solution of 3% w/v benzyl alcohol, 85% w/v nonpolar surfactant Polysorbate 80™, and 65% w/v polyethylene glycol 300, made up to volume in absolute ethanol. The VPD co-solvent system (VPD:D5W) consists of VPD diluted 1:1 with a 5% dextrose in an aqueous solution. This co-solvent system dissolves hydrophobic compounds well, and itself has minimal toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

Alternatively, other delivery systems for hydrophobic pharmaceutical compounds may be employed. Liposomes and emulsions are well-known examples of delivery vehicles or carriers for hydrophobic drugs. Certain organic solvents such as dimethylsulfoxide may also be employed, although usually at the cost of greater toxicity. Additionally, the compounds may be delivered using a sustained-release system, such as semipermeable matrices of solid hydrophobic polymers containing the therapeutic agent. Various sustained-release materials have been developed and are well-known to those skilled in the art. Sustained-release capsules may, depending on their chemical nature, release the compounds for from 2 or 3 weeks up to over 100 days. Depending on the chemical nature and the biological stability of the therapeutic agent, additional strategies for protein stabilization may be employed.

### c) Effective Dosage

Pharmaceutical compositions suitable for use in the present invention include compositions in which the active ingredients are contained in an amount effective to achieve its intended purpose. More specifically, a therapeutically effective amount means an amount of a compound effective to prevent, alleviate or ameliorate symptoms of disease. Determination of a therapeutically effective amount is well within the capability of those skilled in the art, especially in light of the detailed disclosure provided herein.

Many of the compounds of the present invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free acid or base forms.

In the oral pharmaceutical composition, the active ingredient preferably has a crystalline structure with a low degree of crystallinity, which makes it possible to solve the problems associated with poor solubility of naphthoquinone-based compounds of Formula 1 and significantly improve a dissolution rate and in vivo absorption rate.

As used herein, the term “degree of crystallinity” is defined as the weight fraction of the crystalline portion of the total crystalline compound and may be determined by a conventional method known in the alt. For example, measurement of the degree of crystallinity may be carried out by a density method or precipitation method which calculates the crystallinity degree by previous assumption of a preset value obtained by addition and/or reduction of appropriate values to/from each density of the crystalline portion and the amorphous portion, a method involving measurement of the heat of fusion, an X-ray method in which the crystallinity degree is calculated by separation of the crystalline diffraction fraction and the noncrystalline diffraction fraction from X-ray diffraction intensity distribution upon X-ray diffraction analysis, or an infrared method which calculates the crystallinity degree from a peak of the width between crystalline bands of the infrared absorption spectrum.

In the oral pharmaceutical composition, the crystallinity degree of the active ingredient is preferably 50% or less. More preferably, the active ingredient may have an amorphous structure from which the intrinsic crystallinity of the material was completely lost. The amorphous naphthoquinone compound exhibits a relatively high solubility, as compared to the crystalline naphthoquinone compound, and can significantly improve a dissolution rate and in vivo absorption rate of the drug. Further, the amorphous structure enhances solubilization and wettability of the drug per se and effectively supports relatively uniform solubility even under pH fluctuations in vivo, thus maximizing in vivo absorption of the drug and minimizing absorption variation.

In one preferred embodiment of the present invention, the amorphous structure may be formed during preparation of the active ingredient into microparticles or fine particles (micronization of the active ingredient). The microparticles may be prepared, for example by spray drying of active ingredients, melting methods involving formation of melts of active ingredients with polymers, co-precipitation involving formation of co-precipitates of active ingredients with polymers after dissolution of active ingredients in solvents, inclusion body formation, solvent volatilization, or mechanical milling. Preferred is spray drying or mechanical milling.

The spray drying is a method of making fine particles by dissolving an active ingredient in a certain solvent and spray-drying the resulting solution. During the spray-drying process, a high percent of the crystallinity of the naphthoquinone-based compound is lost to thereby result in an amorphous state, and therefore a spray-dried product in the form of a fine powder is obtained.

The mechanical milling is a method of grinding the active ingredient into fine particles by applying strong physical force to active ingredient particles. According to this method, application of high-impact energy results in changes in the crystal geometry of active ingredients, that is, from a crystalline structure to an amorphous structure.

The mechanical milling may be carried out by using a variety of milling processes such as jet milling, ball milling, vibration milling, hammer milling, and the like. Particularly preferred is jet milling which can be carried out using elevated air pressure, at a temperature of less than 40° C.

Meanwhile, a decreasing particle diameter of the particulate active ingredient leads to an increasing specific surface area, thereby increasing the dissolution rate or solubility. However, an excessively small particle diameter makes it difficult to prepare fine particles having such a size and also brings about agglomeration or aggregation of particles which may result in deterioration of the solubility. Therefore, in one preferred embodiment, the particle diameter of the active ingredient may be in a range of 5 nm to 500 μm. In this range, the particle agglomeration or aggregation can be maximally inhibited, and the dissolution rate and solubility can be maximized due to a high specific surface area of the particles.

Preferably, a surfactant may be additionally added to prevent the particle agglomeration or aggregation which may occur during formation of the fine particles, and/or an antistatic agent may be additionally added to prevent the occurrence of static electricity.

If necessary, a moisture-absorbent material may be further added during the milling process. The naphthoquinone-based compound of Formula 1 has a tendency to be crystallized by water, so incorporation of the moisture-absorbent material inhibits recrystallization of the naphthoquinone-based compound over time and enables maintenance of increased solubility of compound particles due to micronization. Further, the moisture-absorbent material serves to suppress coagulation and aggregation of the pharmaceutical composition while not adversely affecting therapeutic effects of the active ingredient.

Examples of the surfactant may include, but are not limited to, anionic surfactants such as docusate sodium and sodium lauryl sulfate; cationic surfactants such as benzalkonium chloride, benzethonium chloride and cetrimide; nonionic surfactants such as glyceryl monooleate, polyoxyethylene sorbitan fatty acid ester, and sorbitan ester; amphiphilic polymers such as polyethylene-polypropylene polymer and polyoxyethylene-polyoxypropylene polymer (Poloxamer), and Gelucire™ series (Gattefosse Corporation, USA); propylene glycol monocaprylate, oleoyl macrogol-6-glyceride, linoleoyl macrogol-6-glyceride, caprylocaproyl macrogol-8-glyceride, propylene glycol monolaurate, and polyglyceryl-6-dioleate. These materials may be used alone or in any combination thereof.

Examples of the moisture-absorbent material may include, but are not limited to, colloidal silica, light anhydrous silicic acid, heavy anhydrous silicic acid, sodium chloride, calcium silicate, potassium aluminosilicate, calcium aluminosilicate, and the like. These materials may be used alone or in any combination thereof.

Some of the above-mentioned moisture absorbents may also be used as the antistatic agent.

The surfactant, antistatic agent, and moisture absorbent are added in a certain amount that is capable of achieving the above-mentioned effects, and such an amount may be appropriately adjusted depending upon micronization conditions. Preferably, the additives may be used in a range of 0.05 to 20% by weight, based on the total weight of the active ingredient.

In one preferred embodiment, during formulation of the pharmaceutical composition into preparations for oral administration, water-soluble polymers, solubilizers and disintegration-promoting agents may be further added. Preferably, formulation of the composition into a desired dosage form may be made by mixing the additives and the particulate active ingredient in a solvent and spray-drying the mixture to obtain a desired product while maintaining an amorphous structure.

The water-soluble polymer is of help to maintain the amorphous state of the active ingredient naphthoquinone-based compound, prevent aggregation of the particulate active ingredients, and render surroundings of naphthoquinone-based compound molecules or particles hydrophilic to consequently enhance water solubility thereof.

Preferably, the water-soluble polymer is a pH-independent polymer, and can bring about crystallinity loss and enhanced hydrophilicity of the active ingredient, even under the between- and within-individual variation of the gastrointestinal pH.

Preferred examples of the water-soluble polymers may include at least one selected from the group consisting of cellulose derivatives such as methyl cellulose, hydroxymethyl cellulose, hydroxyethyl cellulose, ethyl cellulose, hydroxyethylmethyl cellulose, carboxymethyl cellulose, hydroxypropylmethyl cellulose, hydroxypropylmethyl cellulose phthalate, sodium carboxymethyl cellulose, and carboxymethylethyl cellulose; polyvinyl alcohols; polyvinyl acetate, polyvinyl acetate phthalate, polyvinylpyrrolidone or polymers containing the same; polyalkene oxide or polyalkene glycol, and polymers containing the same. Preferred is hydroxypropylmethyl cellulose.

In the pharmaceutical composition, an excessive content of the water-soluble polymer which is higher than a given level provides no further increased solubility, but disadvantageously brings about various problems such as overall increases in the hardness of the formulation, and non-penetration of an eluent into the formulation, by formation of films around the formulation due to excessive swelling of water-soluble polymers upon exposure to the eluent. That is, when an increasing content of the water-soluble polymer reaches a maximum in terms of amorphisation of the naphthoquinone-based compound, the solubility enhancement of the drug due to crystallinity loss thereof also inevitably reaches the uppermost limit. Therefore, in order to further maximize the solubility of the formulation, physical properties of the naphthoquinone-based compound exhibiting partial crystallinity should be modified.

In this respect, the solubilizer serves to enhance solubilization and wettability of the sparingly-soluble naphthoquinone-based compound, and can significantly reduce the bioavailability variation of the naphthoquinone-based compound originating from diets and the time difference of drug administration after dietary uptake. The solubilizer may be selected from conventionally widely used surfactants or amphiphiles, and specific examples of the solubilizer may refer to the surfactants as defined above.

The disintegration-promoting agent serves to improve the drug release rate, and enables rapid release of the drug at the target site to thereby increase bioavailability of the drug.

Preferred examples of the disintegration-promoting agent may include, but are not limited to, at least one selected from the group consisting of Croscarmellose sodium, Crospovidone, calcium carboxymethylcellulose, starch glycolate sodium and lower substituted hydroxypropyl cellulose. Preferred is Croscarmellose sodium.

Upon taking into consideration various factors as described above, it is preferred to add 10 to 1000 parts by weight of the water-soluble polymer, 1 to 30 parts by weight of the disintegration-promoting agent and 0.1 to 20 parts by weight of the solubilizer, based on 100 parts by weight of the active ingredient.

In addition to the above-mentioned ingredients, other materials known in the art in connection with formulation may be optionally added, if necessary.

The solvent for spray drying is a material exhibiting a high solubility without modification of physical properties thereof and easy volatility during the spray drying process. Preferred examples of such a solvent may include, but are not limited to, dichloromethane, chloroform, methanol, and ethanol. These materials may be used alone or in any combination thereof. Preferably, a content of solids in the spray solution is in a range of 5 to 50% by weight, based on the total weight of the spray solution.

According to the experiments conducted by the inventors of the present invention, it was confirmed that the active ingredient exhibited significant increases in the dissolution rate and solubility with maintenance of amorphousness after spray drying, even though the dissolution rate and solubility of the active ingredient were increased to some extent by addition of the water-soluble polymer and the solubilizer prior to spray drying. This is believed to be due to that there is a limit to lower crystallization energy of the active ingredient naphthoquinone-based compound only with solvation effects of these additives. On the other hand, it was also confirmed that a desired level of solubility was not achieved upon spray drying after materials used in conventional formulations were added in addition to the water-soluble polymer and the solubilizer. That is, naphthoquinone-based compounds as in Formula 1 are more preferable when they are formulated with a specific combination of additives in accordance with the present invention and subsequent spray drying.

The oral pharmaceutical composition contains the active ingredient in an amount effective to achieve its intended purpose, that is, therapeutic purpose. More specifically, a therapeutically effective amount refers to an amount of the compound effective to prevent, alleviate or ameliorate symptoms of disease. Determination of the therapeutically effective amount is well within the capability of those skilled in the art, especially in light of the detailed disclosure provided herein.

Further, the oral pharmaceutical composition is particularly effective for the treatment and/or prevention of metabolic diseases, degenerative diseases, and mitochondrial dysfunction-related diseases. Examples of the metabolic diseases may include, but are not limited to, obesity, obesity complications, liver diseases, arteriosclerosis, cerebral apoplexy, myocardial infarction, cardiovascular diseases, ischemic diseases, diabetes, diabetes-related complications and inflammatory diseases.

Complications caused from obesity include, for example hypertension, myocardiac infarction, varicosis, pulmonary embolism, coronary artery diseases, cerebral hemorrhage, senile dementia, Parkinson's disease, type 2 diabetes, hyperlipidemia, cerebral apoplexy, various cancers (such as uterine cancer, breast cancer, prostate cancer, colon cancer and the like), heart diseases, gall bladder diseases, sleep apnea syndrome, arthritis, infertility, venous ulcer, sudden death, fatty liver, hypertrophic cardiomyopathy (HCM), thromboembolism, esophagitis, abdominal wall hernia (Ventral Hernia), urinary incontinence, cardiovascular diseases, endocrine diseases and the like.

Diabetic complications include, for example hyperlipidemia, hypertension, retinopathy, renal insufficiency, and the like.

Examples of the degenerative diseases may include Alzheimer's disease, Parkinson's disease and Huntington's disease.

Diseases arising from mitochondrial dysfunction may include for example, multiple sclerosis, encephalomyelitis, cerebral radiculitis, peripheral neuropathy, Reye's syndrome, Friedrich's ataxia, Alpers syndrome, MELAS, migraine, psychosis, depression, seizure and dementia, paralytic episode, optic atrophy, optic neuropathy, retinitis pigmentosa, cataract, hyperaldosteronemia, hypoparathyroidism, myopathy, amyotrophy, myoglobinuria, muscular hypotonia, myalgia, reduced exercise tolerance, renal tubulopathy, renal failure, hepatic failure, hepatic dysfunction, hepatomegaly, sideroblastic anemia (iron-deficiency anemia), neutropenia, thrombocytopenia, diarrhea, villous atrophy, multiple vomiting, dysphagia, constipation, sensorineural hearing loss (SNHL), mental retardation, epilepsy, and the like.

As used herein, the term “treatment” refers to stopping or delaying of the disease progress, when the drug is used in the subject exhibiting symptoms of disease onset. The term “prevention” refers to stopping or delaying of symptoms of disease onset, when the drug is used in the subject exhibiting no symptoms of disease onset but having high risk of disease onset.

### BRIEF DESCRIPTION OF THE DRAWINGS

FIG. 1 and FIG. 2 graphically show body weight gain (%) and body weight measured in Experimental Example 1;

FIG. 3 graphically shows body weight vs. food uptake changes in mice to which a prodrug composition of the present invention was administered;

FIG. 4 graphically shows GOP/GTP levels in mice to which a prodrug composition of the present invention was administered;

FIG. 5 graphically shows blood levels of T-cholesterol in mice to which a prodrug composition of the present invention was administered;

FIG. 6 graphically shows blood levels of LDL in mice to which a prodrug composition of the present invention was administered;

FIG. 7 graphically shows blood levels of T-bilirubin in mice to which a prodrug composition of the present invention was administered;

FIG. 8 graphically shows blood levels of LDH (Lactate Dehydrogenase) in mice to which a prodrug composition of the present invention was administered; and

FIG. 9 graphically shows blood levels of BUN (Blood Urea Nitrogen) and Creatine in mice to which a prodrug composition of the present invention was administered.

### DETAILED DESCRIPTION OF PREFERRED EMBODIMENTS

Now, the present invention will be described in more detail with reference to the following Examples. These examples are provided only for illustrating the present invention and should not be construed as limiting the scope and spirit of the present invention.

### Example 1

### Synthesis of 5-acetyloxy-2,2-dimethyl-3,4-dihydro-2H-benzo[h]chromen-6-yl N-(t-butoxycarbonyl)isoleucinate

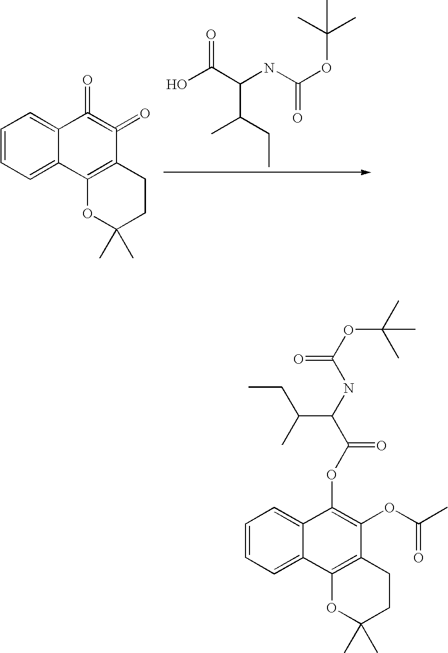

5 g of zinc powder, 5 g of 2,2-dimethyl-3,4-dihydro-2H-benzo[h]chromene-5,6-dione, 16.5 g of Na₂S₂O₄, 8.1 g of N-(t-butoxycarbonyl)isoleucine, 2.8 mL of triethylamine, 17.5 g of HBTU, and 100 mL of DMF were mixed and stirred at room temperature for 15 hours. 300 mL of EtOAc was added to the reaction mixture. The reaction mixture was filtered and washed with water. The organic extract was dried over Na₂SO₄ and concentrated under reduced pressure. The resulting residue was dissolved in 40 mL of acetic anhydride, and 4.0 g of zinc powder and 4.53 g of triethylamine were added thereto. The reaction mixture was heated with vigorous stirring at 85□ for 2 hours and then cooled. The solvent was removed under reduced pressure.

The resulting residue was dissolved in 200 mL of EtOAc and washed with water. The organic extract was dried over Na₂SO₄ and concentrated under reduced pressure. The unpurified product was purified by column chromatography (EtOAc/hexane), such that the final product reached a purity of 60%. 4.1 g (yield: 33%) of the title compound was obtained as a partially pure white solid crystal.

### Example 2

### Synthesis of 5-acetyloxy-2,2-dimethyl-3,4-dihydro-2H-benzo[h]chromen-6-yl Isoleucinate Hydrochloride

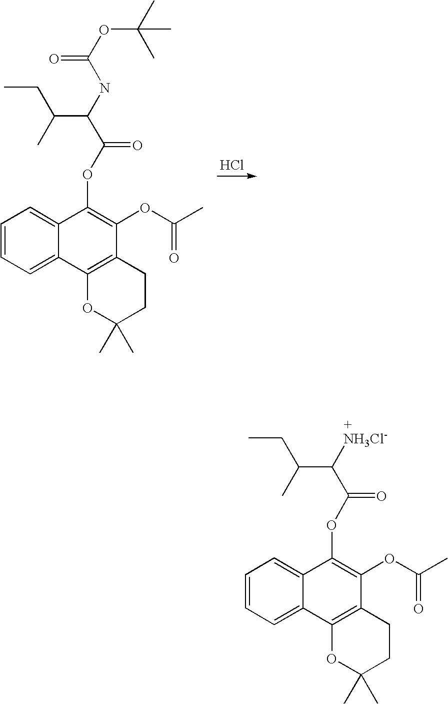

5-(acetyloxy)-2,2-dimethyl-3,4-dihydro-2H-benzo[h]chromen-6-yl N-(t-butoxycarbonyl)isoleucinate prepared in Example 2 was dissolved in 1,4-dioxane to which a solution of hydrogen chloride in anhydrous 1,4-dioxane was then added. The reaction mixture was stirred at room temperature for 6 hours and dried under reduced pressure to afford of the title compound (yield: 98%) as a white solid.

### Example 3

### Synthesis of 2-[5-acetyloxy-2,2-dimethyl-3,4-dihydro-2H-benzo[h]chromen-6-yl]1-t-butyl-carboxyl-imidazole-2-ethylamine-2-carboxylate

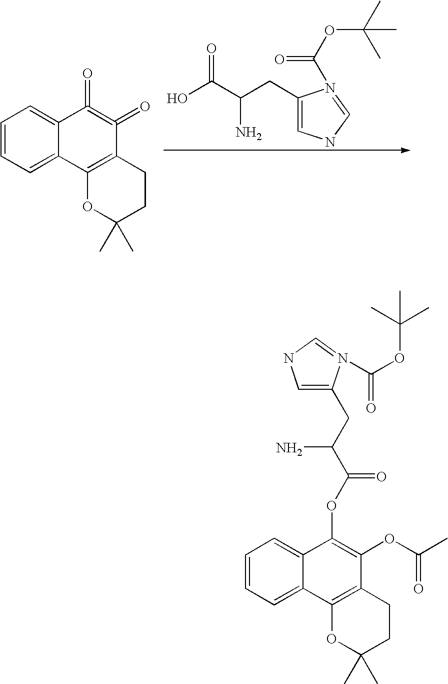

Analogously to Example 1, the title compound (yield: 29%) was prepared as a white solid, except that a mixture of 4 g of zinc powder, 4 g of 2,2-dimethyl-3,4-dihydro-2H-benzo[h]chromene-5,6-dione, 9.5 g of Na₂S₂O₄, 9.2 g of N-(t-butoxycarbonyl)histidine, 3.7 μL of triethylamine, 15.5 g of HBTU and 90 mL of DMF was used, and acetylation was carried out using 2.4 g of zinc powder, 3.9 g of triethylamine and 50 mL of an acetic anhydride.

### Example 4

### Synthesis of 5-acetoxy-2,2-dimethyl-3,4-dihydro-2H-benzo[h]chromen-6-yl L-histidinate Hydrochloride

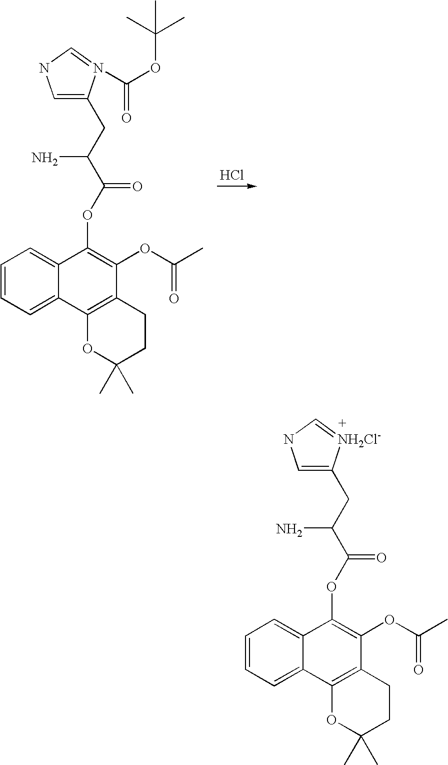

Analogously to Example 2, the title compound (yield: 88%) was prepared as a white solid, except that 2-[5-acetyloxy-2,2-dimethyl-3,4-dihydro-2H-benzo[h]chromen-6-yl]1-t-butyl-carboxyl-imidazole-2-ethylamine-2-carboxylate prepared in Example 3 was used.

### Example 5

### Synthesis of 5,6-diacetoxy-2,2-dimethyl-3,4-dihydro-2H-benzo[h]chromene

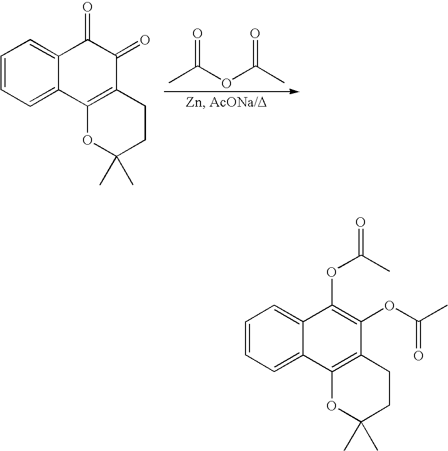

12.0 g of 2,2-dimethyl-3,4-dihydro-2H-benzo[h]chromene-5,6-dione, 16.1 g of zinc powder, 50 mL of anhydrous acetic acid, and 2.0 g of sodium acetate were mixed and vigorously stirred under reflux for 1 hour. The reaction mixture was cooled to room temperature and filtered. The filtered solid was washed once with 200 mL of EtOAc. The filtrate was distilled under reduced pressure to remove anhydrous acetic acid and EtOAc. The residue was dissolved in 200 mL of CH₂Cl₂ and washed with water. The organic extract was dried over Na₂SO₄ and concentrated under reduced pressure. The residue was recrystallized from 150 mL of isopropanol, such that the unpurified final product reached a purity of 99% or higher. 14.8 g (yield: 92%) of the title compound was obtained as a pure white solid crystal.

¹H-NMR (CDCl₃, δ): 8.20 (d, 1H, J=4.5 Hz), 7.67 (d, 1H, J=4.5 Hz), 7.46 (t, 1H, J=4.5 Hz), 7.41 (t, 1H, J=4.5 Hz), 2.67 (t, 1H, J=6.5 Hz), 2.40 (s, 2H), 2.33 (s, 3H), 1.84 (t, 1H, J=6.5 Hz).

### Example 6

### Synthesis of 2,3,3-trimethyl-4,5-diacetoxy-2,3-dihydro-naphtho[1,2-b]furan

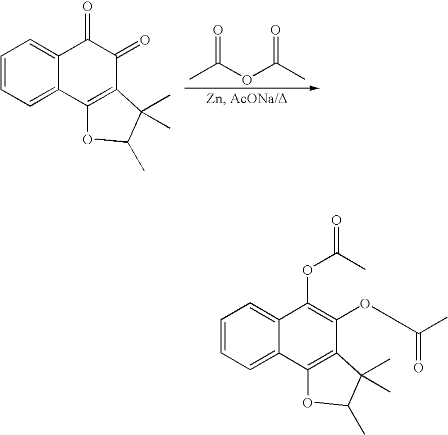

Analogously to Example 5, 15.3 g (yield: 95%) of the title compound was obtained as a pure white solid crystal, except that 12.0 g of 2,3,3-trimethyl-2,3-dihydro-naphtho[1,2-b]furan-4,5-dione was used instead of 2,2-dimethyl-3,4-dihydro-2H-benzo[h]chromene-5,6-dione.

¹H-NMR (CDCl₃, δ): 7.95 (dd, 1H, J=8.8, 1.5 Hz), 7.65 (dd, 1H, J=7.4, 1.5 Hz), 7.39-7.48 (m, 2H), 4.58 (q, 1H, J=6.5 Hz), 2.41 (s, 3H), 2.37 (s, 3H), 1.46 (d, 3H, J=6.5 Hz), 1.40 (s, 3H), 1.20 (s, 3H).

### Example 7

### Synthesis of 2-methyl-4,5-diacetoxy-2,3-dihydro-naphtho[1,2-b]furan

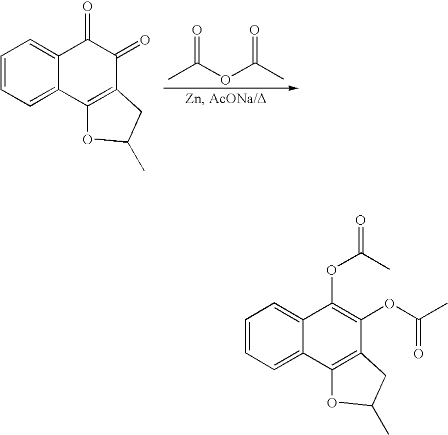

Analogously to Example 5, 15.7 g (yield: 93%) of the title compound was obtained as a pure white solid crystal, except that 12.0 g of 2-methyl-2,3-dihydro-naphtho[1,2-b]furan-4,5-dione was used instead of 2,2-dimethyl-3,4-dihydro-2H-benzo[h]chromene-5,6-dione.

¹H-NMR (CDCl₃, δ): 7.92 (dd, 11, J=7.4, 1.5 Hz), 7.72 (dd, 1H, J=7.7, 1.5 Hz), 7.40-7.48 (m, 2H), 5.18 (m, 1H), 3.40 (dd, 1H, J=15.2, 9.2 Hz), 2.92 (dd, 1H, J=15.2, 7.8 Hz), 2.43 (s, 3H), 2.33 (s, 3H), 1.56 (d, 3H, J=6.2 Hz).

### Example 8

### Synthesis of 5,6-dipropionoxy-2,2-dimethyl-3,4-dihydro-2H-benzo[h]chromene

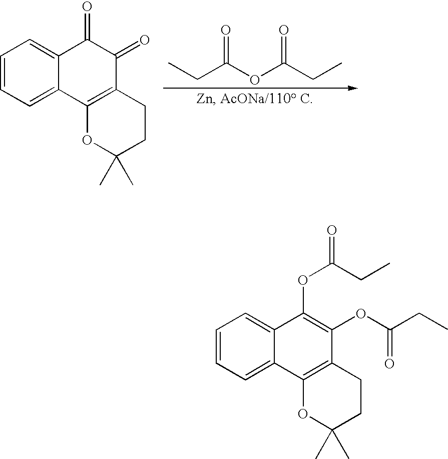

12.0 g of 2,2-dimethyl-3,4-dihydro-2H-benzo[h]chromene-5,6-dione, 16.1 g of zinc powder, 70 mL of anhydrous propionic acid, and 2.0 g of sodium acetate were mixed and vigorously stirred at 110□ for 1 hour. The reaction mixture was cooled to room temperature and filtered. The filtered solid was washed once with 200 mL of EtOAc. The filtrate was distilled under reduced pressure to remove anhydrous propionic acid and EtOAc. The residue was dissolved in 200 mL of CH₂Cl₂ and washed with water. The organic extract was dried over Na₂SO₄ and concentrated under reduced pressure. The residue was recrystallized from 200 mL of isopropanol, such that the unpurified final product reached a purity of 99% or higher. 12.2 g (yield: 86%) of the title compound was obtained as a pure white solid crystal.

¹H-NMR (CDCl₃, δ): 8.22 (dd, 1H, J=7.6, 1.5 Hz), 7.67 (dd, 1H, J=7.8, 1.5 Hz), 7.42-7.50 (m, 2H), 2.72 (q, 2H, J=7.6 Hz), 2.68 (t, 1H, 6.7 Hz), 2.64 (q, 1H, J=7.6 Hz), 1.88 (t, 1H, J=6.7 Hz), 1.43 (s, 6H), 1.37 (t, 3H, J=7.6 Hz), 1.33 (t, 3H, J=7.6 Hz).

### Example 9

### Synthesis of 2,3,3-trimethyl-4,5-dipropionoxy-2,3-dihydro-naphtho[1,2-b]furan

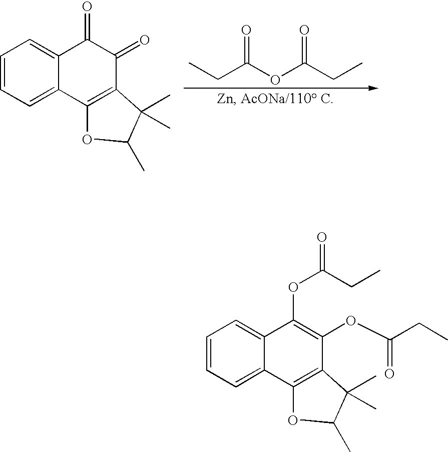

Analogously to Example 8, 12.6 g (yield: 72%) of the title compound was obtained as a pure white solid crystal, except that 12.0 g of 2,3,3-trimethyl-2,3-dihydro-naphtho[1,2-b]furan-4,5-dione was used instead of 2,2-dimethyl-3,4-dihydro-2H-benzo[h]chromene-5,6-dione.

¹H-NMR (CDCl₃, δ): 7.96 (dd, 1H, J=8.8, 1.5 Hz), 7.65 (dd, 1H, J=7.4, 1.5 Hz), 7.40-7.48 (m, 2H), 4.60 (q, 1H, J=6.6 Hz), 2.71 (q, 2H, J=7.6 Hz), 2.66 (q, 1H, J=7.6 Hz), 1.47 (d, 3H, J=6.6 Hz), 1.40 (s, 3H), 1.35 (t, 3H, J=7.6 Hz), 1.34 (t, 3H, J=7.6 Hz), 1.20 (s, 3H).

### Example 10

### Synthesis of 2-methyl-4,5-dipropionoxy-2,3-dihydro-naphtho[1,2-b]furan

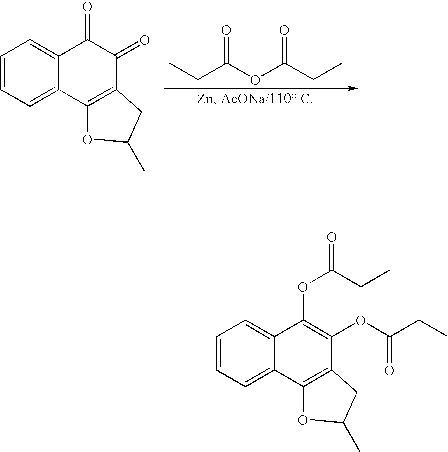

Analogously to Example 8, 14.0 g (yield: 92%) of the title compound was obtained as a pure white solid crystal, except that 10.0 g of 2-methyl-2,3-dihydro-naphtho[1,2-b]furan-4,5-dione was used instead of 2,2-dimethyl-3,4-dihydro-2H-benzo[h]chromene-5,6-dione.

¹H-NMR (CDCl₃, δ): 7.94 (dd, 1H, J=7.3, 1.5 Hz), 7.72 (dd, 1H, J=7.5, 1.5 Hz), 7.41-7.49 (m, 2H), 5.19 (m, 1H), 3.40 (dd, 1H, J=15.2, 9.1 Hz), 2.92 (dd, 1H, J=15.2, 7.8 Hz), 2.74 (q, 2H, J=7.6 Hz), 2.61 (q, 1H, J=7.6 Hz), 1.58 (d, 3H, J=6.2 Hz), 1.38 (t, 3H, J=7.6 Hz), 1.31 (t, 3H, J=7.6 Hz).

### Example 11

### Synthesis of 5,6-dibutanoyloxy-2,2-dimethyl-3,4-dihydro-2H-benzo[h]chromene

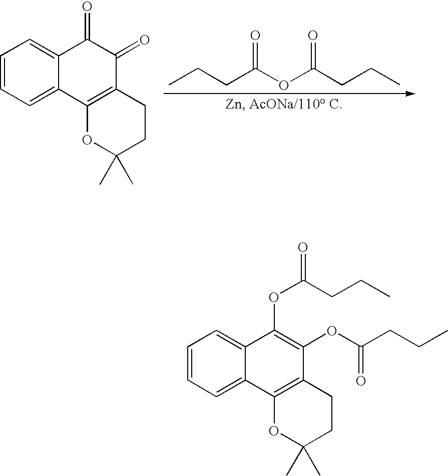

12.0 g of 2,2-dimethyl-3,4-dihydro-2H-benzo[h]chromene-5,6-dione, 16.1 g of zinc powder, 80 mL of anhydrous butanoic acid, and 2.0 g of sodium acetate were mixed and vigorously stirred at 110□ for 1 hour. The reaction mixture was cooled to room temperature and filtered. The filtered solid was washed once with 200 mL of EtOAc. The filtrate was distilled under reduced pressure to remove anhydrous butanoic acid and EtOAc. The residue was dissolved in 200 mL of CH₂Cl₂ and washed with water. The organic extract was dried over Na₉SO₄ and concentrated under reduced pressure. The residue was purified using silica gel to afford 17.6 g (yield: 92%) of the title compound as a high-viscosity transparent liquid compound.

¹H-NMR (CDCl₃, δ): 8.22 (dd, 1H, J=7.6, 1.5 Hz), 7.67 (dd, 1H, J=7.8, 1.5 Hz), 7.42-7.50 (m, 2H), 2.68 (t, 4H, J=7.3 Hz), 2.60 (1,2H, J=7.4 Hz), 1.78-1.92 (m, 4H), 1.83 (t, 2H, J=7.4 Hz), 1.44 (s, 6H), 1.11 (t, 3H, J=7.4 Hz), 1.09 (t, 3H, J=7.4 Hz).

### Example 12

### Synthesis of 2,3,3-trimethyl-4,5-dibutanoyloxy-2,3-dihydro-naphtho[1,2-b]furan

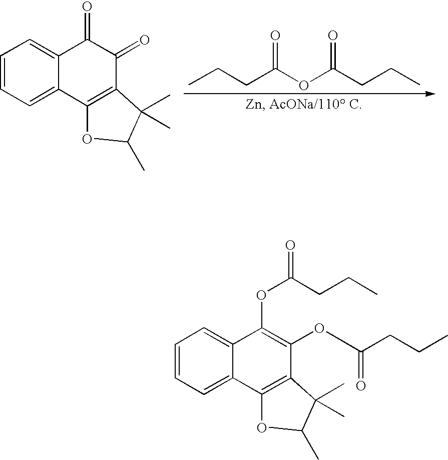

Analogously to Example 11, 12.6 g (yield: 72%) of the title compound was obtained as a high-viscosity transparent liquid compound, except that 12.0 g of 2,3,3-trimethyl-2,3-dihydro-naphtho[1,2-b]furan-4,5-dione was used instead of 2,2-dimethyl-3,4-dihydro-2H-benzo[h]chromene-5,6-dione.

¹H-NMR (CDCl₃, δ): 7.96 (dd, 1H, J=7.2, 1.5 Hz), 7.64 (dd, 1H, J=7.4, 1.5 Hz), 7.40-7.48 (m, 2H), 4.60 (q, 1H, J=6.6 Hz), 2.67 (t, 2H, J=7.3 Hz), 2.61 (t, 2H, J=7.3 Hz), 1.78-1.92 (m, 4H), 1.47 (d, 3H, J=6.6 Hz), 1.41 (s, 3H), 1.20 (s, 3H), 1.11 (t, 3H, J=7.4 Hz), 1.09 (t, 3H, J=7.4 Hz).

### Example 13

### Synthesis of 2-methyl-4,5-dibutanoyloxy-2,3-dihydro-naphtho[1,2-b]furan

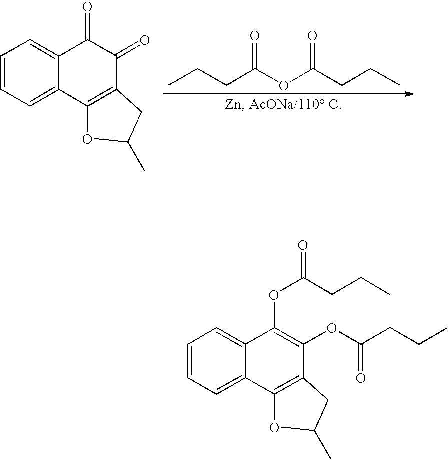

Analogously to Example 11, 16.1 g (yield: 97%) of the title compound was obtained as a transparent liquid compound, except that 10.0 g of 2-methyl-2,3-dihydro-naphtho[1,2-b]furan-4,5-dione was used instead of 2,2-dimethyl-3,4-dihydro-2H-benzo[h]chromene-5,6-dione. The product was pure without further purification.

¹H-NMR (CDCl₃, δ): 7.94 (dd, 1H, J=7.3, 1.5 Hz), 7.72 (dd, 1H, J=7.5, 1.5 Hz), 7.40-7.48 (m, 2H), 5.19 (m, 1H), 3.40 (dd, 1H, J=15.2, 9.1 Hz), 2.92 (dd, 1H, J=15.2, 7.8 Hz), 2.69 (t, 2H, J=7.3 Hz), 2.57 (t, 1H, J=7.3 Hz), 1.90 (m, 2H), 1.81, (m, 2H), 1.56 (d, 3H, J=6.3 Hz), 1.12 (t, 3H, J=7.4 Hz), 1.08 (t, 3H, J=7.4 Hz).

### Example 14

### Synthesis of 10,11-diacetoxy-2,6,6-trimethyl-1,2,6,7,8,9-hexahydro-phenanthro[1,2b]furan

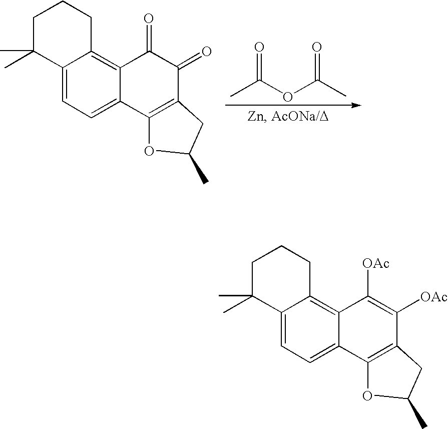

14.7 g of Cryptotanshinone, 16.1 g of zinc powder, 100 mL of anhydrous acetic acid, and 2.0 g of sodium acetate were mixed and vigorously stirred under reflux for 1 hour. The reaction mixture was cooled to room temperature and filtered. The filtered solid was washed once with 200 mL of EtOAc. The filtrate was distilled under reduced pressure to remove anhydrous acetic acid and EtOAc. The residue was dissolved in 300 mL of CH₂Cl₂ and washed with water. The organic extract was dried over Na₂SO₄ and concentrated under reduced pressure. The residue was recrystallized from 350 mL of isopropanol, such that the unpurified final product was purified to a purity of 99% or higher. 16.8 g (yield: 88%) of the title compound was obtained as a pure white solid crystal.

¹H-NMR (CDCl₃, δ): 7.72 (d, 1H, J=8.6 Hz), 7.40 (d, 1H, J=8.6 Hz), 4.85 (t, 1H, J=8.8 Hz), 4.29 (dd, 1H, J=7.4, 6.4 Hz), 3.7 (m, 1H), 3.14 (b, 2H), 2.36 (s, 3H), 2.33 (s, 3H), 1.78 (m, 2H), 1.65 (m, 2H), 1.31 (s, 3H), 1.31 (s, 3H), 1.30 (d, 3H).

### Experimental Example 1

### Weight Loss Effects of Naphthoquinone-Based Compounds in Obese Mice (ob/ob)

4-week-old C57BL/6 male mice were purchased from Orient BIO Inc. (Korea). Animals were raised for 10 weeks in a breeding room maintained at a temperature of 23° C., 55% humidity, illumination of 300 to 500 lux, a 12-h light/dark (L/D) cycle, and ventilation of 10 to 18 times/hr. Experiments included animals weighing 45 kg or higher. Animals were fed ad libitum a high-fat diet (45 kcal % from fat, D12451, Research Diet) and tap water as drinking water.

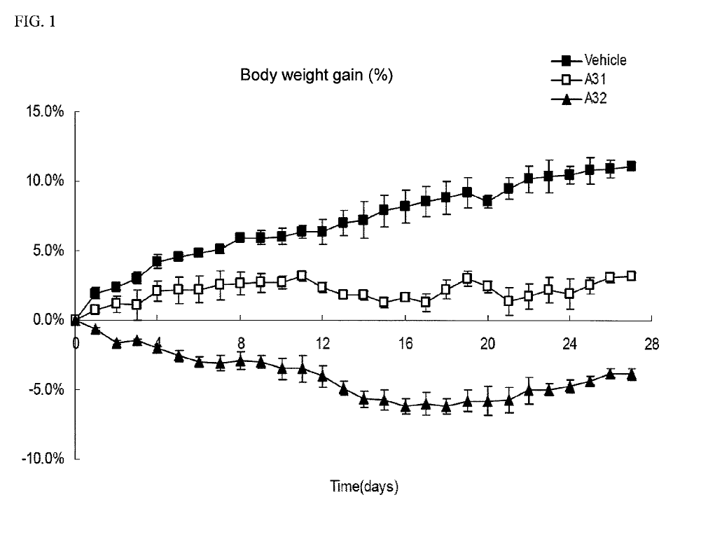

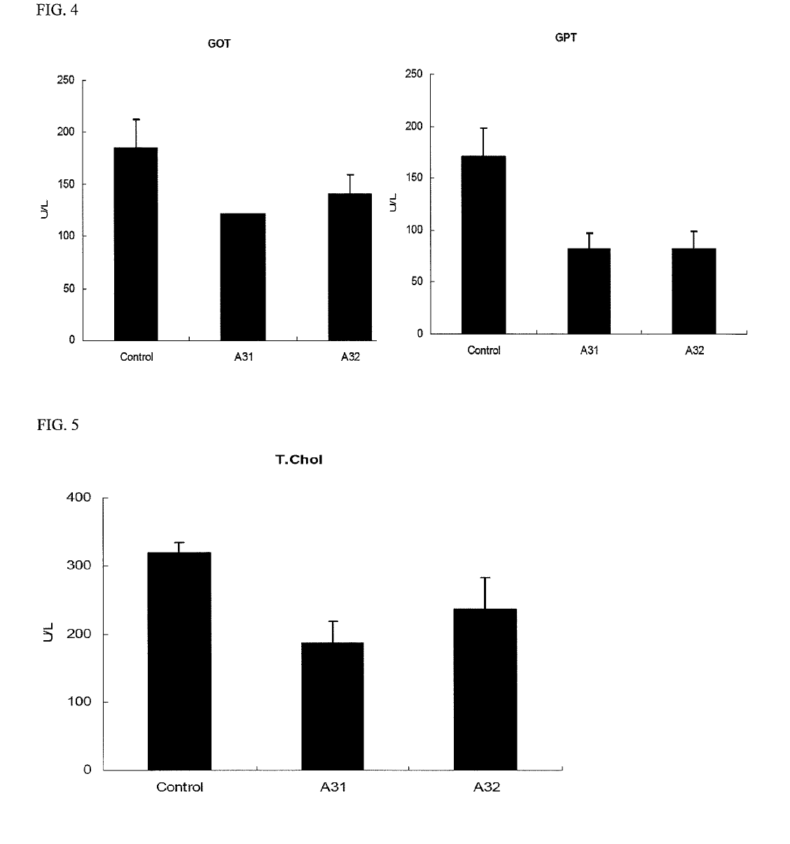

Each group of animals was administered 195 mg/kg of the prodrug compound of Example 14 (hereinafter often simply referred to as “A31”) and 65 mg/kg of the prodrug compound of Example 5 (hereinafter often simply referred to as “A32”) for 4 weeks, as set forth in Table 1 below. Observations were made on changes in body weight and food intake, with respect to a time course of administration. The results obtained are shown in FIGS. 1 to 3. After administration was complete, biochemical indices were assayed including changes in adipose tissue distribution, hepatic functions, and lipid and enzyme levels. The results obtained are shown in FIGS. 4 to 7.

TABLE 1

| Group | Dose (mg/Kg) | Others (head/route/period) |
|---|---|---|
| Control (Vehicle) | Sterile water | Two/oral/28 days |
| Example 14 (A 31) | 195 mg/kg | Three/oral/28 days |
| Example 5 (A 32) | 65 mg/kg | Three/oral/28 days |

FIGS. 1 to 3 graphically show body weight gain (%) and time-course changes of body weight and food intake in mice to which the prodrug composition of the present invention was administered.

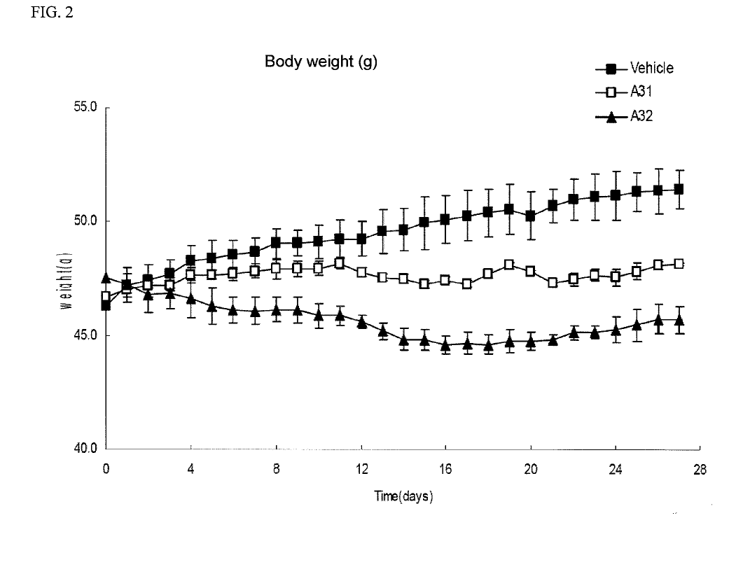

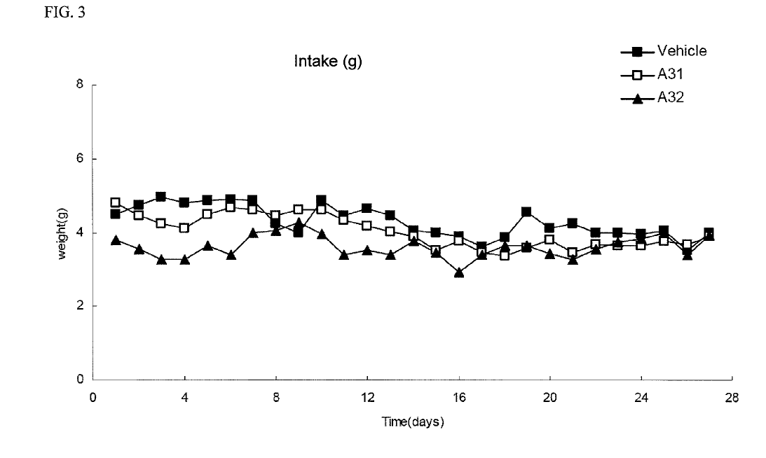

As can be seen from FIGS. 1 and 2, administration of the compound of Example 14 according to the present invention resulted in significant reductions in body weight (g) and body weight gain (%), as compared to the control group. In particular, the animal group with administration of the compound of Example 5 exhibited a negative (−) value of body weight gain, thus representing that the compound of Example 5 has excellent weight-loss effects. Referring to FIG. 3, animals did not exhibit a significant difference in food intake, despite loss of the body weight, as compared to the control group. Therefore, it can be seen that when the prodrug composition of the present invention was administered, in vivo metabolism according to dietary intake is activated to thereby exhibit excellent weight-loss effects.

### Experimental Example 2

### Biochemical Indices of Naphthoquinone-Based Compounds in C57BL/6 Mice

FIGS. 4 and 5 show changes of biochemical indices in the blood of C57BL/6 mice to which the prodrug composition of Example 5 and the prodrug composition of Example 14 were administered. As compared to the control group with reference to FIG. 4, the prodrug-administered groups exhibited decreased blood levels of GOT/GTP enzymes which are contained in hepatocytes and are released into the blood stream to result in elevated blood levels thereof when hepatocytes are disrupted or permeability of the hepatocyte membrane is increased.

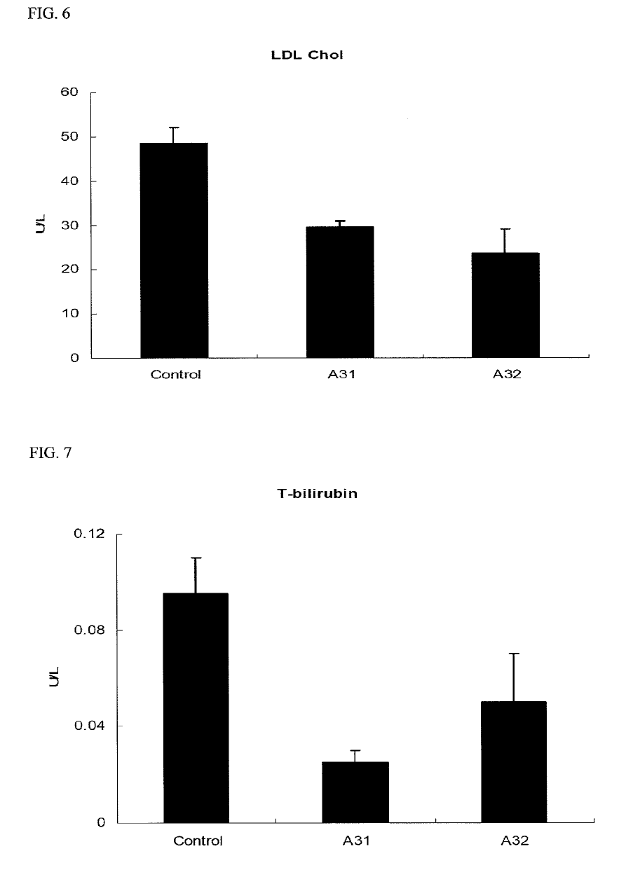

Referring to FIG. 5, a blood level of T-cholesterol, known as a risk factor of arteriosclerosis (particularly in coronary and cerebral arteries), was much lower in the group with administration of the prodrug composition of the present invention, as compared to the control group. Further, it is known that when HDL is elevated and LDL is lowered in a clinical atherogenic index=HDL/LDL, it is possible to ameliorate arteriosclerosis. In this connection, it was confirmed, as shown in FIG. 6, that the experimental group treated with the prodrug composition of the present invention exhibited a significantly lowered LDL value, as compared to the control group. From these results, it can be seen that the prodrug composition of the present invention is effective for the preparation of anti-arteriosclerotic agents, due to lowering effects of a blood lipid level.

FIGS. 7 and 8 show blood levels of T-bilirubin and lactate dehydrogenase (LDH) measured respectively in the experimental group treated with the prodrug composition of the present invention and in the control group. As is generally known, T-bilirubin is produced with release of hemoglobin according to breakdown of red blood cells when hepatic functions are deteriorated. Referring to FIG. 7, it can be seen that a blood level of T-bilirubin was significantly decreased when the prodrug composition of the present invention was treated. Further, it was confirmed that an LDH value of FIG. 8 exhibiting high activity in hepatic diseases, cardiac diseases and hematological diseases was also definitely decreased as compared to the control group.

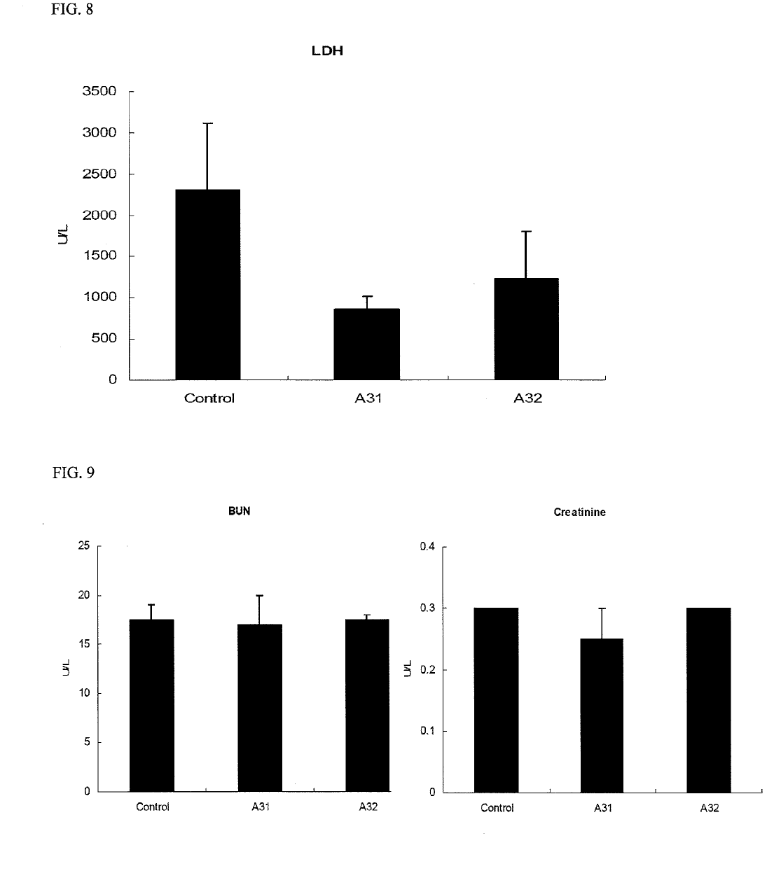

FIG. 9 shows blood levels of Blood Urea Nitrogen (BUN) and Creatine measured respectively in the experimental group treated with the prodrug composition of the present invention and the control group. Referring to FIG. 9, there was no noticeable difference in BUN and Creatine values correlated with the protein metabolism, between the prodrug composition-administered experimental group and the control group. That is, it can be seen that the prodrug composition of the present invention plays an effective role in normalization of the protein metabolism.

### INDUSTRIAL APPLICABILITY

As apparent from the foregoing, the present invention enables effective manufacture of a medicament for treatment or prevention of metabolic syndrome diseases, by increasing in vivo solubility and activity of poorly soluble naphthoquinone-based compounds as well as by improving bioavailability and subsequently pharmacokinetic properties thereof.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A use of a prodrug composition comprising a naphthoquinone-based compound represented by Formula 1 below, for the manufacture of a medicament for treatment or prevention of metabolic syndrome diseases: 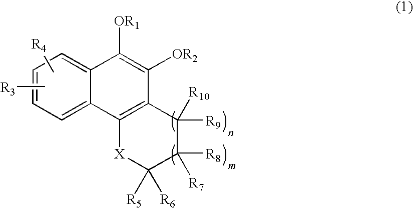
wherein,
A) X is selected from the group consisting of C, N, O and S;
B) R₁ and R₂ are each independently —SO₃—Na⁺, or a substituent represented by Formula 2 below or a salt thereof: 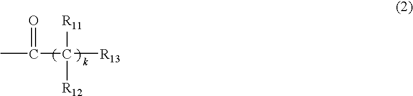
wherein
R₁₁ and R₁₂ are each independently hydrogen or substituted or unsubstituted, linear or branched C₁-C₂₀ alkyl;
R₁₃ is selected from the group consisting of substituents i) to viii):
i) hydrogen;
ii) substituted or unsubstituted, linear or branched C₁-C₂₀ alkyl;
iii) substituted or unsubstituted amine;
iv) substituted or unsubstituted C₃-C₁₀ cycloalkyl or C₃-C₁₀ heterocycloalkyl;
v) substituted or unsubstituted C₄-C₁₀ aryl or C₄-C₁₀ heteroaryl;
vi) —(CRR′—NR″CO)₁—R₁₄ wherein R, R′ and R″ are each independently hydrogen or substituted or unsubstituted, linear or branched C₁-C₂₀ alkyl, R₁₄ is selected from the group consisting of hydrogen, substituted or unsubstituted amine, cycloalkyl, heterocycloalkyl, aryl and heteroaryl, and 1 is in the range of 1 to 5;
vii) substituted or unsubstituted carboxyl; and
viii) —OSO₃—Na⁺; and
k is in the range of 0 to 20, provided that when k is 0, R₁₁ and R₁₂ are absent and R₁₃ is directly bonded to a carbonyl group;
C) R₃ and R₄ are each independently hydrogen, halogen, alkoxy, hydroxy or C₁-C₆ allyl, or R₃ and R₄ may be taken together to form a cyclic structure;
D) R₅, R₆, R₇, R₈, R₉, and R₁₀ are each independently selected from the group consisting of hydrogen, hydroxy, and C₁-C₂₀ alkyl, C₁-C₂₀ alkene, C₁-C₂₀ alkoxy, C₃-C₁₀ cycloalkyl, C₃-C₁₀ heterocycloalkyl, C₄-C₁₀ aryl, C₄-C₁₀ heteroaryl and any combination thereof, any of which groups being substituted or unsubstituted, or two of R₅ to R₁₀ may be taken together to form a cyclic structure; and
E) m and n are each independently 0 or 1, provided that when either of m and n is 0, carbon atoms adjacent to m or n may form a cyclic structure via a direct bond.

2. The method according to claim 1, wherein R₂ is a structure of —CO—(CH₂)₀₋₂₀—R₁₄ wherein R₁₄ is selected from the group consisting of hydrogen, hydroxy, carboxyl, C₄-C₁₀ aryl, C₄-C₁₀ heteroaryl, C₃-C₁₀ cycloalkyl, C₃-C₁₀ heterocycloalkyl, amine and any combination thereof.

3. The method according to claim 1, wherein k is in the range of 0 to 3.

4. The method according to claim 1, wherein R₁ and R₂ are each independently a substituent represented by Formula 3:
—CO—(CRR′—NR″CO)ᵢ—R₁₄ (3)
wherein R, R′ and R″ are each independently hydrogen or substituted or unsubstituted, linear or branched C₁-C₂₀ alkyl or R and R″ may be taken together to form a cyclic structure, R₁₄ is selected from the group consisting of hydrogen, substituted or unsubstituted C₁-C₁₀ alkyl, substituted or unsubstituted C₁-C₁₀ alkoxy, substituted or unsubstituted amine, substituted or unsubstituted C₃-C₁₀ cycloalkyl, heterocycloalkyl, aryl and heteroaryl, and i is in the range of 1 to 3.

5. The method according to claim 4, wherein R₁ or R₂ contains one or more amino acids selected from the group consisting of glycine, alanine, valine, leucine, isoleucine, phenylalanine, tryptophan, methionine, serine, threonine, cysteine, tyrosine, asparagine, glutamine, aspartic acid, glutamic acid, lysine, arginine, proline and histidine.

6. The method according to claim 5, wherein the amino acid is glycine, alanine, valine, proline, isoleucine or histidine.

7. The method according to claim 1, wherein when R₁₃ is substituted carboxyl, the substituent is C₁-C₂₀ alkyl which is optionally substituted with one or more substituents selected from the group consisting of hydrogen, hydroxy, carboxyl, C₄-C₁₀ aryl and C₄-C₁₀ heteroaryl.

8. The method according to claim 1, wherein when the aryl or heteroaryl is substituted, the substituent is selected from the group consisting of amine, C₁-C₆ alkyl, C₄-C₁₀ aryl, C₄-C₁₀ heteroaryl, C₃-C₁₀ cycloalkyl, C₃-C₁₀ heterocycloalkyl and any combination thereof.

9. The method according to claim 8, wherein the aryl or heteroaryl is selected from the group consisting of pyridine, C₄-C₁₀ aryl or C₄-C₁₀ heteroaryl substituted with C₁-C₆ alkyl, and aryl substituted with C₁-C₆ alkyl and/or C₄-C₁₀ aryl, or C₄-C₁₀ heteroaryl.

10. The method according to claim 1, wherein when cycloalkyl or heterocycloalkyl is substituted, the substituent is selected from the group consisting of hydroxy, C₁-C₆ alkyl, C₁-C₆ alkoxy, C₄-C₁₀ aryl, C₄-C₁₀ heteroaryl, C₃-C₁₀ cycloalkyl, C₃-C₁₀ heterocycloalkyl and any combination thereof.

11. The method according to claim 10, wherein the heterocycloalkyl is selected from the group consisting of piperidine, piperazine and pyrrolidine which is optionally substituted with C₁-C₆ alkyl.

12. The method according to claim 1, wherein X is O.

13. The method according to claim 1, wherein R₃ and R₄ are each independently hydrogen.

14. The method according to claim 1, wherein m is 1, n is 0, and R₃, R₄ and R₆ are each independently hydrogen, or R₃, R₄ and R₈ are each independently hydrogen.

15. The method according to claim 1, wherein m is 1, n is 1, and R₃, R₄, R₇, R₈, R₉ and R₁₀ are each independently hydrogen.

16. The method according to claim 1, wherein an active ingredient in the medicament has a crystallinity degree of 50% or less.

17. The method according to claim 16, wherein the active ingredient has an amorphous structure.

18. The method according to claim 17, wherein the amorphous structure is formed during preparation of the active ingredient into microparticles.

19. The method according to claim 18, wherein the microparticles have a particle diameter of 5 nm to 500 μn.

20. The method according to claim 18, wherein the microparticles are formed by spray drying or mechanical milling of the active ingredient.

21. The method according to claim 20, wherein the mechanical milling is carried out by jet milling.

22. The method according to claim 18, wherein micronization of the active ingredient is carried out with addition of one or more materials selected from the group consisting of a surfactant, an antistatic agent and a moisture-absorbent material.

23. The method according to claim 22, wherein the surfactant is at least one selected from the group consisting of anionic surfactants such as docusate sodium and sodium lauryl sulfate; cationic surfactants such as benzalkonium chloride, benzethonium chloride and cetrimide; nonionic surfactants such as glyceryl monooleate, polyoxyethylene sorbitan fatty acid ester, and sorbitan ester; amphiphilic polymers such as polyethylene-polypropylene polymer and polyoxyethylene-polyoxypropylene polymer (Poloxamer), and Gelucire™ series; propylene glycol monocaprylate, oleoyl macrogol-6-glyceride, linoleoyl macrogol-6-glyceride, caprylocaproyl macrogol-8-glyceride, propylene glycol monolaurate, and polyglyceryl-6-dioleate.

24. The method according to claim 22, wherein the moisture-absorbent material is at least one selected from the group consisting of colloidal silica, light anhydrous silicic acid, heavy anhydrous silicic acid, sodium chloride, calcium silicate, potassium aluminosilicate, and calcium aluminosilicate.

25. The method according to claim 1, wherein the metabolic syndrome disease is at least one selected from the group consisting of obesity, diabetes, arteriosclerosis, hypertension, hyperlipidemia, liver diseases, cerebral apoplexy, myocardial infarction, ischemic diseases, and cardiovascular diseases.
